(19) United States
(12) Patent Application Publication
(10) Pub. No.: US 2025/0188042 A1
(43) Pub. Date: Jun. 12, 2025

(54) HETEROCYCLIC COMPOUND, PREPARATION METHOD THEREFOR, AND USE THEREOF

(71) Applicant: SICHUAN UNIVERSITY, Chengdu, Sichuan (CN)

(72) Inventors: Jiang LIU, Chengdu, Sichuan (CN); Yao YUAN, Chengdu, Sichuan (CN); Hang ZHAO, Chengdu, Sichuan (CN); Kaichao WANG, Chengdu, Sichuan (CN); Shaojuan SONG, Chengdu, Sichuan (CN); Qianming CHEN, Chengdu, Sichuan (CN)

(21) Appl. No.: 18/846,086

(22) PCT Filed: Jan. 10, 2024
(86) PCT No.: PCT/CN2024/071475

§ 371 Date: Sep. 11, 2024

(30) Foreign Application Priority Data
Jan. 10, 2023 (CN) 202310035826.0

(51) Int. Cl. C07D 239/54; A61K 31/513; A61K 31/52; A61K 31/522; A61P 35/00; C07D 239/36; C07D 239/47; C07D 239/553; C07D 239/557; C07D 239/60; C07D 473/04; C07D 473/16; C07D 473/18; C07D 473/30; C07D 473/34; C07D 473/40

(52) U.S. Cl. CPC C07D 239/54; A61K 31/513; A61K 31/52; A61K 31/522; A61P 35/00; C07D 239/36; C07D 239/47; C07D 239/553; C07D 239/557; C07D 239/60; C07D 473/04; C07D 473/16; C07D 473/18; C07D 473/30; C07D 473/34; C07D 473/40

(57) ABSTRACT

A heterocyclic compound, a preparation method therefor, and a use thereof are provided. The structure of the heterocyclic compound is as shown in formula (I). The compound can effectively inhibit the proliferation of squamous cell carcinoma, and provides a new choice for preparing drugs for preventing and/or treating squamous cell carcinoma.

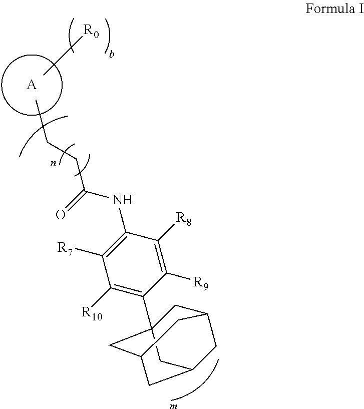

### FIELD OF THE INVENTION

The present invention belongs to the field of chemical medicines, and specifically relates to a novel heterocyclic compound, preparation methods therefor, and uses thereof.

### BACKGROUND OF THE INVENTION

Malignant oral and maxillofacial tumors are common malignant tumors in the head and neck regions, with over 90% being oral squamous cell carcinoma (OSCC), which is the eighth largest malignant tumor worldwide. OSCC is usually detected and diagnosed relatively late, with high rates of early metastasis and postoperative recurrence, poor prognosis, and other characteristics, causing that the mortality rate of OSCC remains high. 90% of early OSCC patients can be cured with surgical treatment, but among diagnosed patients, early cases are less than 10%. For advanced OSCC, chemotherapy is generally used first, and then surgical treatment may be considered after tumor shrinkage.

Platinum chemotherapeutic medicaments are the first-line medicaments for the treatment of OSCC. However, chemotherapy resistance greatly limits the clinical application of platinum chemotherapeutic medicaments, and the development of new medicaments that can effectively treat OSCC is of great significance.

### CONTENT OF THE INVENTION

The object of the present invention is to provide a novel heterocyclic compound, preparation methods therefor, and uses thereof.

The present invention provides the compounds represented by formula I, pharmaceutically acceptable salts thereof, and stereoisomers thereof:

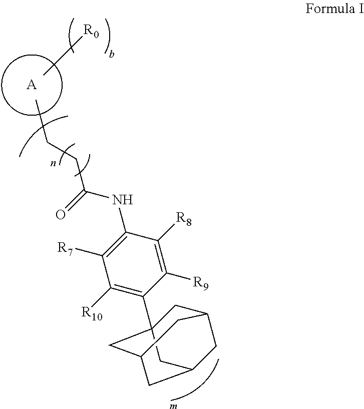

wherein, ring A is selected from N-containing fused rings or 5-6-membered unsaturated N-containing heterocycles;
b is an integer selected from 0 to 6;
R₀ is each independently selected from the group consisting of ═O, ═S, H, amino, halogen, C₁₋₆ alkyl, C₁₋₆ alkoxy, hydroxyl, carboxyl, cyano, alkylamine, heterocycle-substituted alkyl, (aromatic ring)-substituted alkyl;
m is an integer selected from 1 to 3; n is an integer selected from 0 to 5; R₇, R₈, R₉, and R₁₀ are each independently selected from the group consisting of H, amino, halogen, C₁₋₆ alkyl, C₁₋₆ alkoxy, halogen, hydroxyl, and carboxyl.
Further, the structure of the compound is as represented by formula II:

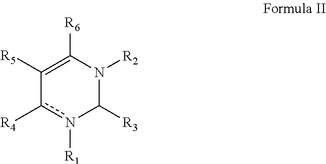

wherein, [symbol] represents [symbol] or [symbol] ;

R₁ is selected from the group consisting of absence, H or

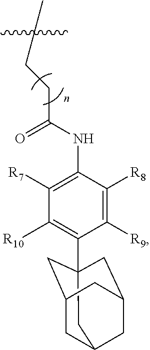

R₂ is selected from the group consisting of H or

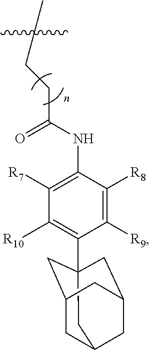

and provided that R₁ is absence or H, R₂ is not H;

n is an integer selected from 0 to 3; R₇, R₈, R₀, and R₁₀ are each independently selected from the group consisting of H, amino, halogen, C₁₋₄ alkyl, C₁₋₄ alkoxy, halogen, hydroxyl, and carboxyl;

R₃ is selected from the group consisting of ═O, ═S, H, amino, halogen, C₁₋₄ alkyl, C₁₋₄ alkoxy, hydroxyl, and carboxyl;

R₄ is selected from the group consisting of ═O, ═S, H, amino, halogen, C₁₋₄ alkyl, C₁₋₄ alkoxy, hydroxyl, and carboxyl;

and at least one of R₃ and R₄ is ═O or ═S;

R₅ is selected from the group consisting of H, amino, halogen, C₁₋₄ alkyl, C₁₋₄ alkoxy, hydroxyl, carboxyl, cyano, alkylamine, heterocycle-substituted alkyl, and (aromatic ring)-substituted alkyl;

R₆ is selected from the group consisting of H, amino, halogen, C₁₋₄ alkyl, C₁₋₄ alkoxy, hydroxyl, and carboxyl.

Further, the structure of the compound is as represented by formula II-x:

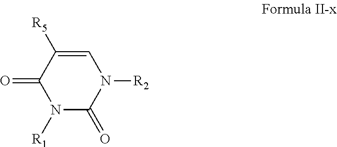

wherein, R₅ is selected from the group consisting of H, amino, halogen, C₁₋₂ alkyl, C₁₋₂ alkoxy, hydroxyl, carboxyl, cyano, alkylamine, heterocycle-substituted alkyl, and (aromatic ring)-substituted alkyl;

R₁ is selected from the group consisting of H or

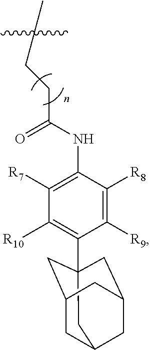

R₂ is selected from the group consisting of H or

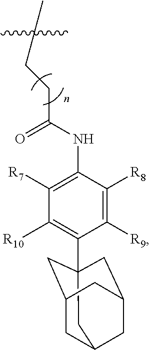

and R₁ and R₂ are not both H; n is an integer selected from 0 to 3; R₇, R₈, R₉, and R₁₀ are each independently selected from the group consisting of H, amino, halogen, C₁₋₄ alkyl, C₁₋₄ alkoxy, halogen, hydroxyl, and carboxyl.

Further, the structure of the compound is as represented by formula II-a or formula II-b:

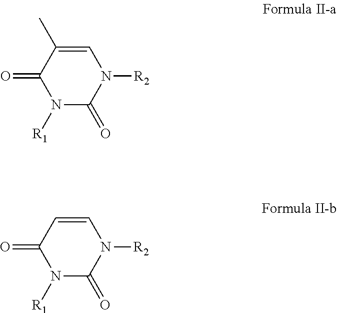

wherein, R₁ is selected from the group consisting of H or

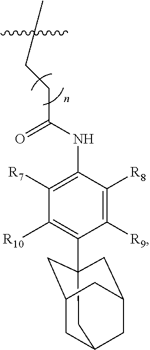

R₂ is selected from the group consisting of H or

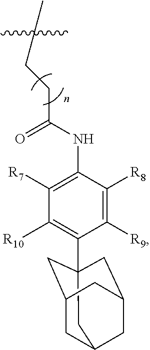

and R₁ and R₂ are not both H; n is an integer selected from 0 to 3; R₇, R₈, R₉, and R₁₀ are each independently selected from the group consisting of H, amino, halogen, C₁₋₄ alkyl, C₁₋₄ alkoxy, halogen, hydroxyl, and carboxyl.

Further, the structure of the compound is as represented by formula II-c or formula II-d:

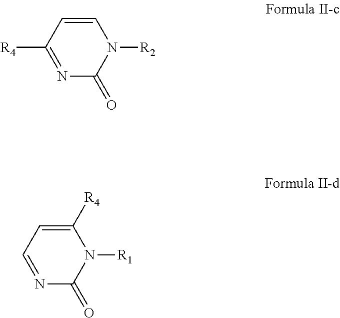

wherein, R₁ is

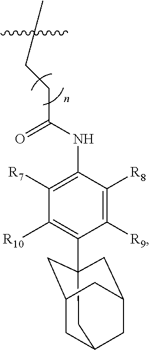

### R₂ is

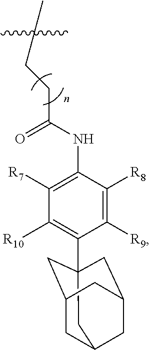

n is an integer selected from 0 to 3; R₇, R₈, R₉, and R₁₀ are each independently selected from the group consisting of H, amino, halogen, C₁₋₄ alkyl, C₁₋₄ alkoxy, halogen, hydroxyl, and carboxyl;

R₄ is selected from the group consisting of H, amino, halogen, C₁₋₄ alkyl, C₁₋₄ alkoxy, hydroxyl, and carboxyl.

Further, the structure of the compound is as represented by formula III-a or formula III-b:

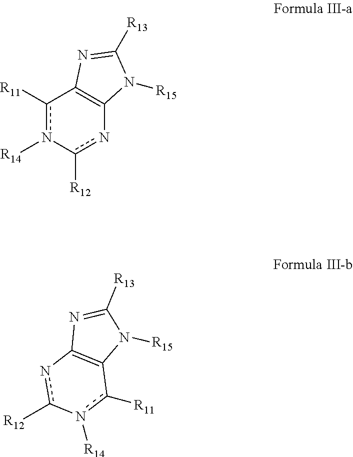

wherein, [symbol] represents [symbol] or [symbol] ;

R₁₄ is selected from the group consisting of absence, H, or

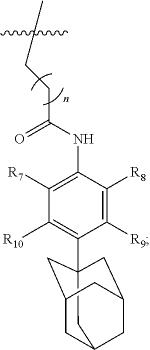

R₁₅ is selected from the group consisting of H or

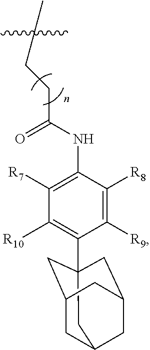

and provided that R₁₄ is absence or H, R₁₅ is not H;

n is an integer selected from 0 to 3; R₇, R₈, R₉, and R₁₀ are each independently selected from the group consisting of H, C₁₋₄ alkyl, C₁₋₄ alkoxy, halogen, hydroxyl, and carboxyl;

R₁₁ is selected from the group consisting of ═O, ═S, H, amino, halogen, C₁₋₄ alkyl, C₁₋₄ alkoxy, hydroxyl, and carboxyl;

R₁₂ is selected from the group consisting of ═O, ═S, H, amino, halogen, C₁₋₄ alkyl, C₁₋₄ alkoxy, hydroxyl, and carboxyl;

R₁₃ is selected from the group consisting of H, amino, halogen, C₁₋₄ alkyl, C₁₋₄ alkoxy, hydroxyl, and carboxyl.

Further, the structure of the compound is as represented by formula III-c or formula III-d:

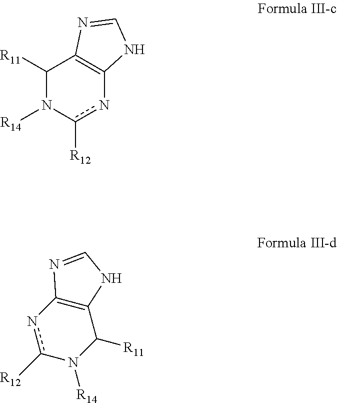

wherein, R₁₄ is

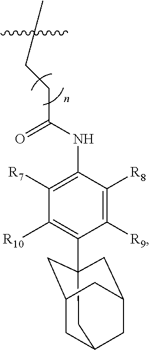

n is an integer selected from 0 to 3; R₇, R₈, R₉, and R₁₀ are each independently selected from the group consisting of H, C1-4 alkyl, C1-4 alkoxy, halogen, hydroxyl, and carboxyl;

R₁₁ is selected from the group consisting of ═O, ═S, H, amino, halogen, C₁₋₄ alkyl, C₁₋₄ alkoxy, hydroxyl, and carboxyl;

R₁₂ is selected from the group consisting of ═O, ═S, H, amino, halogen, C₁₋₄ alkyl, C₁₋₄ alkoxy, hydroxyl, and carboxyl.

Further, the compound is selected from the group consisting of:

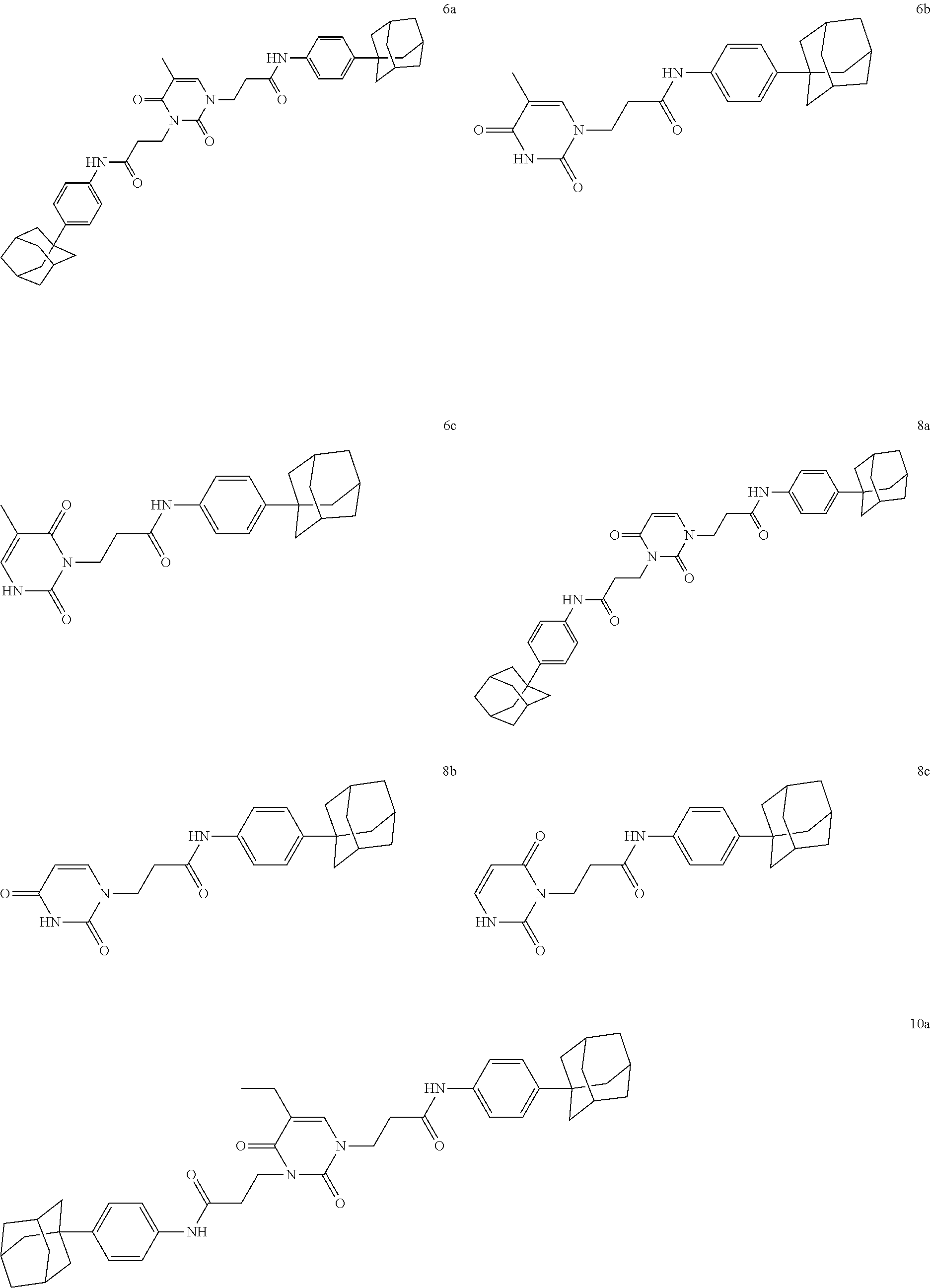

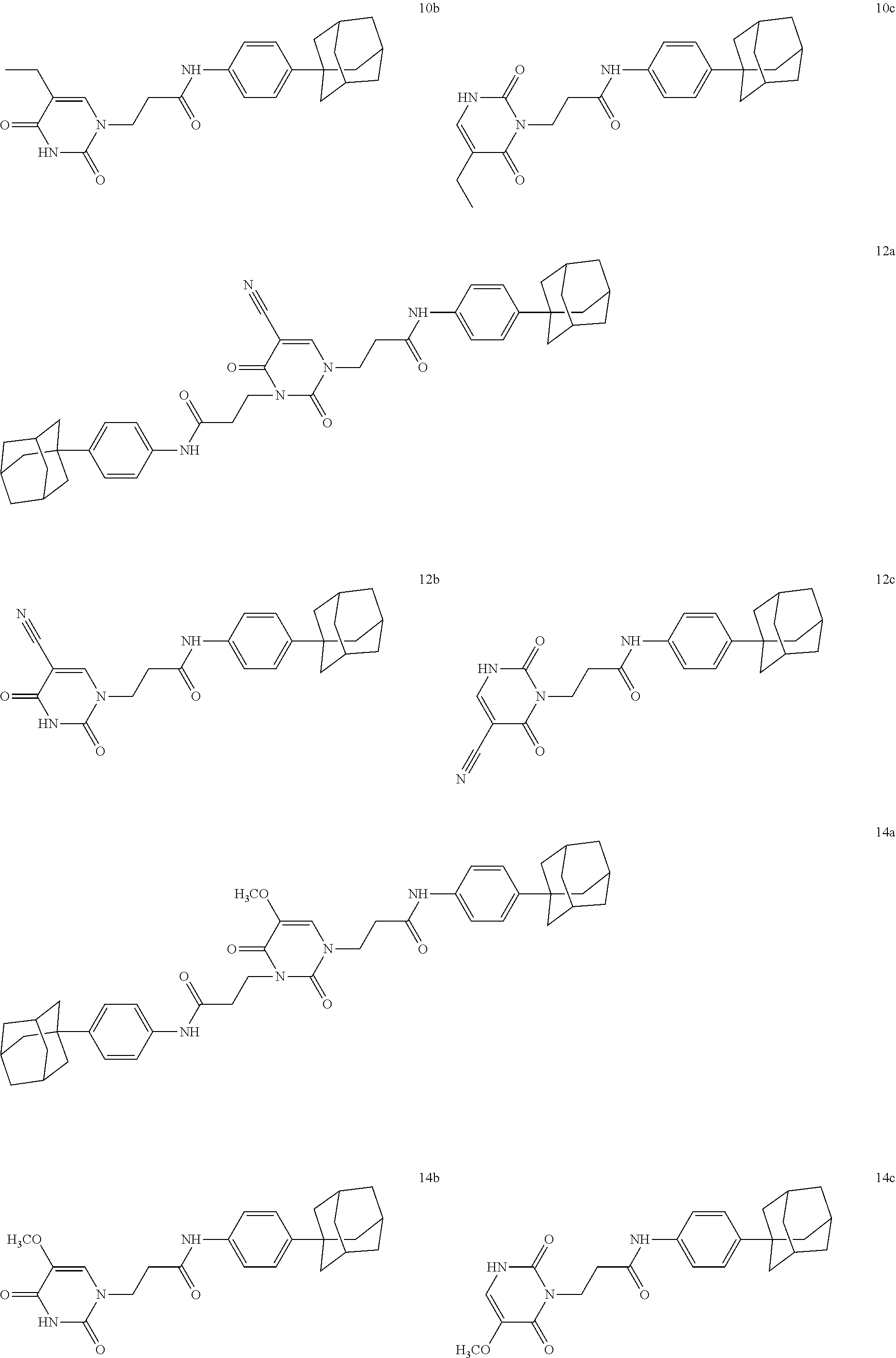

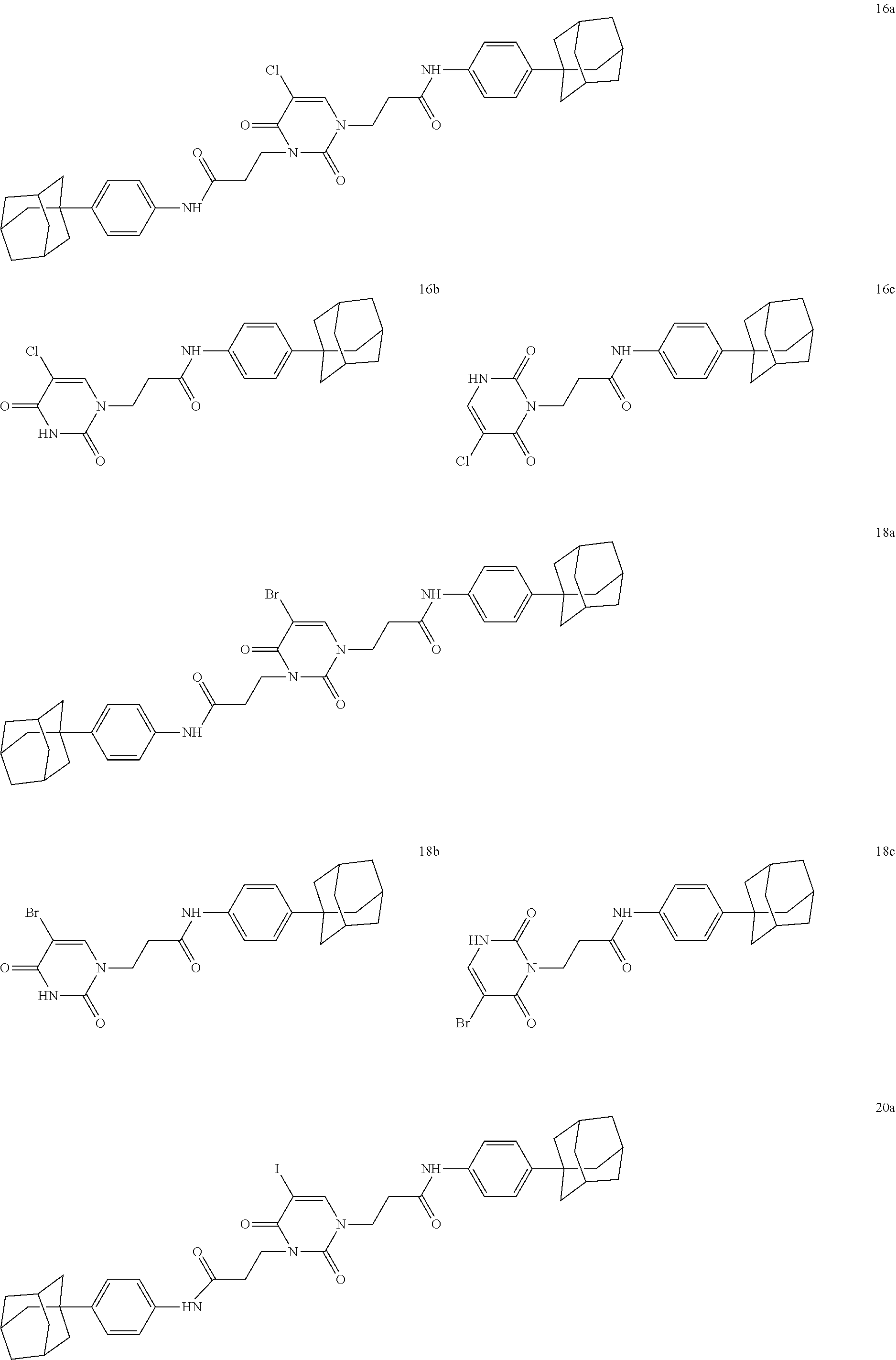

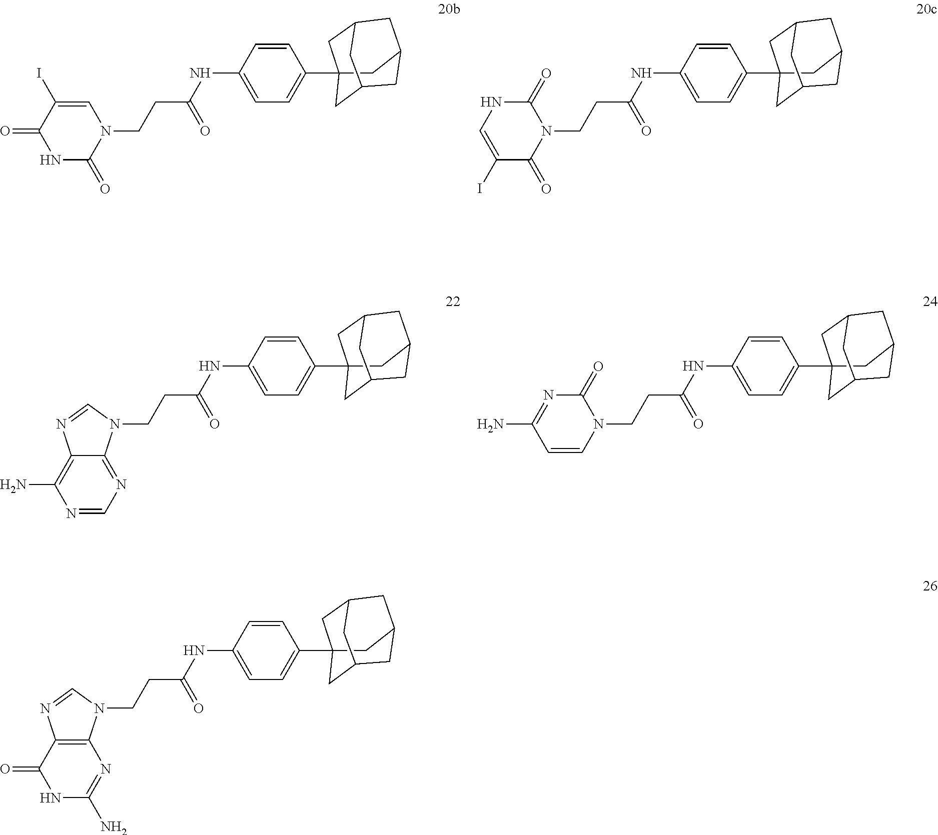 [SMILES: O=C(CCn1cc(I)c(=O)[nH]c1=O)Nc1ccc(C23CC4CC(CC(C4)C2)C3)cc1 ; O=C(CCn1c(=O)[nH]cc(I)c1=O)Nc1ccc(C23CC4CC(CC(C4)C2)C3)cc1 ; Nc1ncnc2c1ncn2CCC(=O)Nc1ccc(C23CC4CC(CC(C4)C2)C3)cc1 ; Nc1ccn(CCC(=O)Nc2ccc(C34CC5CC(CC(C5)C3)C4)cc2)c(=O)n1 ; Nc1nc2c(ncn2CCC(=O)Nc2ccc(C34CC5CC(CC(C5)C3)C4)cc2)c(=O)[nH]1]

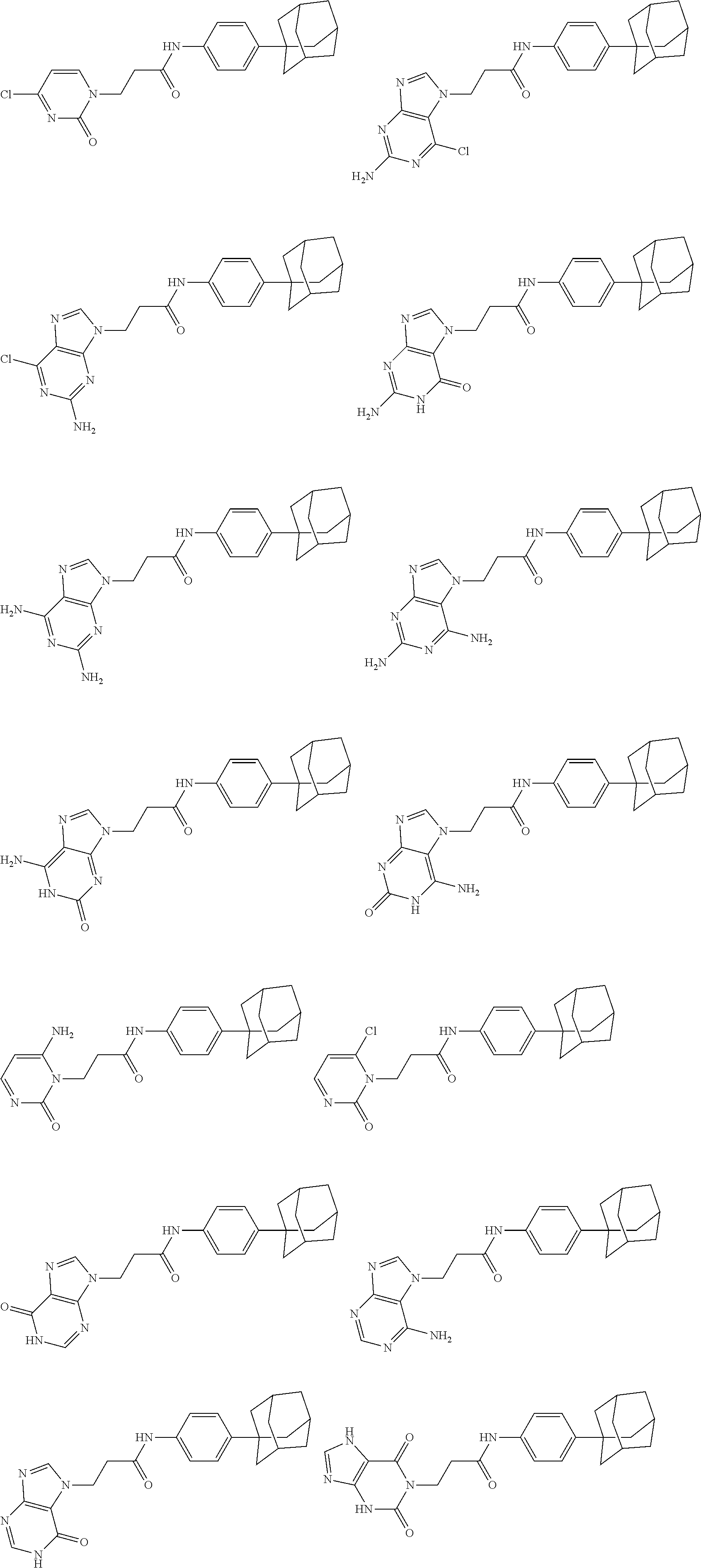

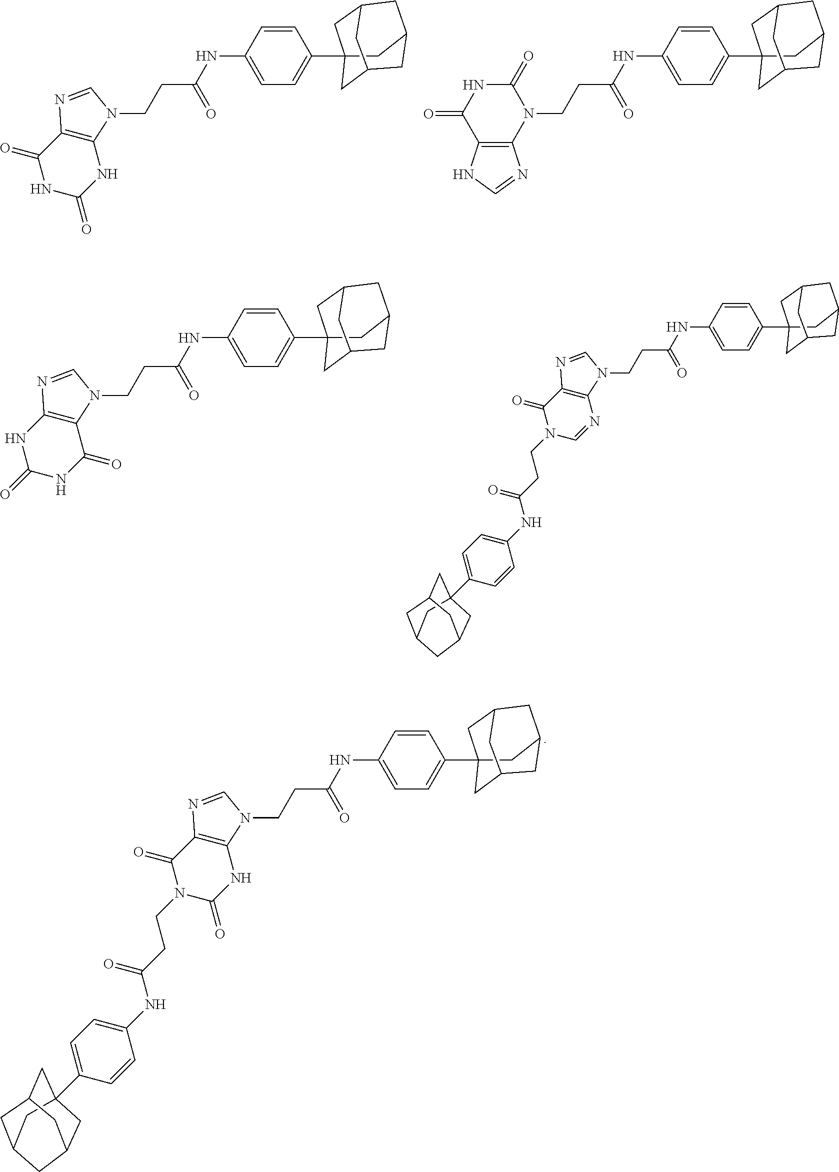 [SMILES: O=C(CCn1cnc2c(=O)[nH]c(=O)[nH]c21)Nc1ccc(C23CC4CC(CC(C4)C2)C3)cc1 ; O=C(CCn1c(=O)[nH]c(=O)c2[nH]cnc21)Nc1ccc(C23CC4CC(CC(C4)C2)C3)cc1 ; O=C(CCn1cnc2[nH]c(=O)[nH]c(=O)c21)Nc1ccc(C23CC4CC(CC(C4)C2)C3)cc1 ; O=C(CCn1cnc2c(ncn2CCC(=O)Nc2ccc(C34CC5CC(CC(C5)C3)C4)cc2)c1=O)Nc1ccc(C23CC4CC(CC(C4)C2)C3)cc1 ; O=C(CCn1c(=O)[nH]c2c(ncn2CCC(=O)Nc2ccc(C34CC5CC(CC(C5)C3)C4)cc2)c1=O)Nc1ccc(C23CC4CC(CC(C4)C2)C3)cc1]

The present invention also provides an anti-tumor pharmaceutical composition, which is a preparation formed by using the above compound as the active ingredient, in combination with pharmaceutically acceptable excipients.

The present invention also provides the use of the above compound in the manufacture of anti-tumor medicaments.

Further, the tumor is squamous cell carcinoma (SCC).

Further, said SCC is head and neck squamous cell carcinoma (HNSCC).

Further, said HNSCC is oral squamous cell carcinoma (OSCC).

Further, said OSCC is tongue squamous cell carcinoma (TSCC).

For the definition of the terms used in the present invention: unless indicated otherwise, the initial definition provided for the group or the term herein is applicable to those in the whole specification; for terms not specifically defined herein, according to the disclosure content and the context, the term will have their common meaning as understood by one of ordinary skill in the art to which this invention pertains.

The minimum and the maximum for the content of carbon atoms in hydrocarbon groups are represented by prefixes, for example, the prefix C_{a-b} alkyl denotes any alkyl containing “a”-“b” carbon atoms. For example, C₁₋₆ alkyl refers to straight or branched alkyls containing 1-6 carbon atoms.

“5-6-membered unsaturated N-containing heterocycle” refers to N-containing unsaturated cyclic hydrocarbon substituents containing 5-6 ring atoms.

“Fused heterocyclic ring” refers to a polycyclic heterocyclic group, in which two rings share two adjacent carbon atoms or heteroatoms.

“N-containing fused heterocyclic ring” refers to a fused heterocyclic ring in which a ring atom is N. For example,

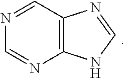

Halogen refers to fluorine, chlorine, bromine, or iodine.

The experimental results indicate that the compounds provided in the present invention can effectively inhibit the proliferation of SCC, and thus provide a new option for the manufacture of medicaments for preventing and/or treating SCC.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1: The inhibitory activities of compounds 6a, 6b, and 6c on the proliferation of squamous cell carcinoma cells, as well as the IC₅₀ value of compound 6a against squamous cell carcinoma cells.

FIG. 2: The inhibitory activities of compounds 8a, 8b, and 8c on the proliferation of squamous cell carcinoma cells, as well as the IC₅₀ value of compound 8a against squamous cell carcinoma cells.

FIG. 3: The inhibitory activities of compounds 10a, 10b, and 10c on the proliferation of squamous cell carcinoma cells, as well as the IC₅₀ value of compound 10a against squamous cell carcinoma cells.

FIG. 4: The inhibitory activities of compounds 12a, 12b, and 12c on the proliferation of squamous cell carcinoma cells, as well as the IC₅₀ value of compound 12a against squamous cell carcinoma cells.

FIG. 5: The inhibitory activities of compounds 14a, 14b, and 14c on the proliferation of squamous cell carcinoma cells, as well as the IC₅₀ value of compound 14a against squamous cell carcinoma cells.

FIG. 6: The inhibitory activities of compounds 16a, 16b, and 16c on the proliferation of squamous cell carcinoma cells, as well as the IC₅₀ value of compound 16a against squamous cell carcinoma cells.

FIG. 7: The inhibitory activities of compounds 18a, 18b, and 18c on the proliferation of squamous cell carcinoma cells, as well as the IC₅₀ value of compound 18a against squamous cell carcinoma cells.

FIG. 8: The inhibitory activities of compounds 20a, 20b, and 20c on the proliferation of squamous cell carcinoma cells, as well as the IC₅₀ value of compound 20a against squamous cell carcinoma cells.

FIG. 9: The inhibitory activities of compounds 22, 24, and 26 on the proliferation of squamous cell carcinoma cells, as well as the IC₅₀ values of compounds 22 and 24 against squamous cell carcinoma cells.

### EXAMPLES

The starting materials and equipment used in the present invention were all known products and could be obtained by purchasing those commercially available.

### Example 1: Preparation of Compounds 6a, 6b, and 6c

### 1. Preparation of Compound 4

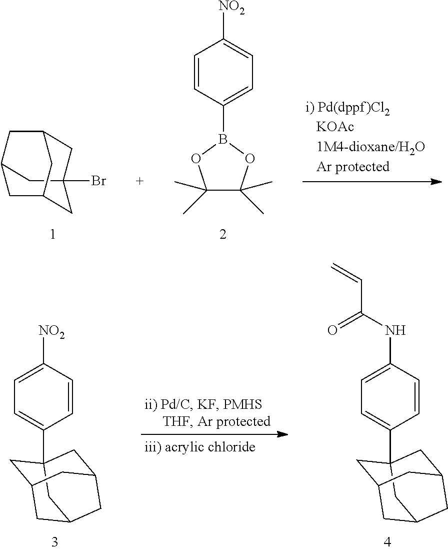

1-bromoadamantane 1 (1 mmol) was dissolved in 10 mL mixed solution of 1,4-dioxane and water, and then the system was purged with argon and stirred at room temperature. Subsequently, excess 4-nitrophenylboronic acid pinacol ester 2 (2 mmol), potassium acetate (3 mmol), and catalytic amount of [1,1′-bis (diphenylphosphino) ferrocene]dichloropalladium (0.04 mmol) were added, and then the mixture was allowed to react under refluxing and argon protection. Once the reaction was completed by TLC detection, the argon protection was removed, and the reaction was quenched with water. The aqueous phase was extracted three times with dichloromethane. The organic phases were combined, and dried over anhydrous Na₂SO₄. The organic solvent was removed by rotatory evaporation under reduced pressure. The crude product of compound 3 obtained was directly used in the next step without further purification.

Compound 3 (1 mmol) obtained in the previous step was dissolved in 10 mL of anhydrous tetrahydrofuran, to which were added catalytic amount of Pd/C (0.05 mmol), excess potassium fluoride (2 mmol), and poly (methylhydrosiloxane) (4 mmol). The reaction was stirred at room temperature under argon protection, and monitored by TLC until compound 3 disappeared. Then, excess acryloyl chloride (2 mmol) was added to the reaction solution, and the reaction was further monitored by TLC until completion. Then, the argon protection was removed, and then the reaction was quenched with water. The aqueous phase was extracted three times with ethyl acetate. The organic phases were combined, and dried over anhydrous Na₂SO₄. The organic solvent was removed by rotatory evaporation under reduced pressure. The residue was isolated and purified by flash column chromatography, to obtain compound 4, with a total yield of 50% for two steps.

Analytic data of compound 4: ¹H NMR (DMSO-d₆, 600 MHZ) δ 10.05 (s, 1H), 7.60-7.56 (m, 2H), 7.31-7.27 (m, 2H), 6.42 (dd, J=17.0, 10.2 Hz, 1H), 6.24 (dd, J=17.0, 2.0 Hz, 1H), 5.72 (dd, J=10.1, 2.0 Hz, 1H), 2.04 (p, J=3.4 Hz, 3H), 1.83 (d, J=3.1 Hz, 6H), 1.76-1.69 (m, 6H). LRMS calcd for C₁₉H₂₃NO [M+Na]⁺ 304.17, found 304.17.

### 2. Preparation of Compounds 6a, 6b, and 6c

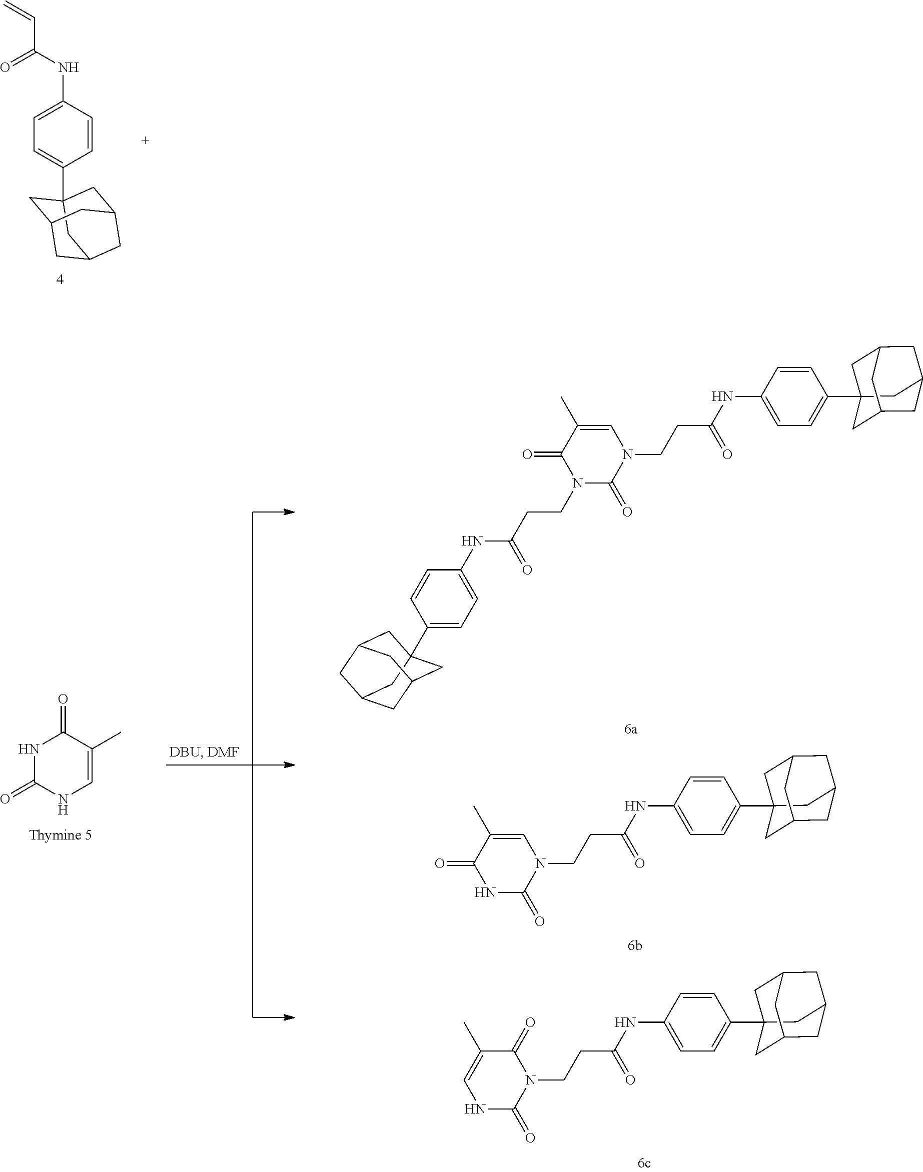

Equal amounts of thymine 5 and 1,8-diazabicyclo [5.4.0]undec-7-ene (DBU), together with excess compound 4, were dissolved in N,N-dimethylformamide (DMF), and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compounds 6a, 6b, and 6c in sequence, with a total yield of 85% to 95%.

Analytic data of compound 6a: ¹H NMR (DMSO-d₆, 600 MHz) δ 9.90 (s, 1H), 9.86 (s, 1H), 7.57 (d, J=1.2 Hz, 1H), 7.48-7.45 (m, 4H), 7.27-7.24 (m, 4H), 4.10 (t, J=7.6 Hz, 2H), 3.96 (t, J=6.7 Hz, 2H), 2.69 (t, J=6.7 Hz, 2H), 2.54 (t, J=7.7 Hz, 2H), 2.05-1.99 (m, 6H), 1.81 (d, J=3.2 Hz, 12H), 1.78 (s, 3H), 1.75-1.67 (m, 12H). LRMS calcd for C₄₃H₅₂N₄O₄ [M+Na]⁺ 711.39, found 711.39.

Analytic data of compound 6b: ¹H NMR (DMSO-d₆, 600 MHz) δ 11.24 (s, 1H), 9.92 (s, 1H), 7.47 (d, J=1.2 Hz, 1H), 7.47-7.44 (m, 2H), 7.28-7.24 (m, 2H), 3.90 (t, J=6.7 Hz, 2H), 2.67 (t, J=6.7 Hz, 2H), 2.03 (dt, J=6.4, 3.2 Hz, 3H), 1.82 (d, J=3.2 Hz, 6H), 1.75-1.68 (m, 9H). LRMS calcd for C₂₄H₂₉N₃O₃ [M+K]⁺ 446.18, found 446.18.

Analytic data of compound 6c: ¹H NMR (DMSO-d₆, 600 MHz) δ 10.91 (s, 1H), 9.86 (s, 1H), 7.49-7.43 (m, 2H), 7.31 (s, 1H), 7.28-7.23 (m, 2H), 4.06 (t, J=7.7 Hz, 2H), 2.54 (t, J=7.7 Hz, 2H), 2.04 (dt, J=6.4, 3.4 Hz, 3H), 1.83 (d, J=3.3 Hz, 6H), 1.78 (d, J=1.2 Hz, 3H), 1.75-1.69 (m, 6H). LRMS calcd for C₂₄H₂₉N₃O₃ [M−H]⁻ 406.21, found 406.21.

### Example 2: Preparation of Compounds 8a, 8b, and 8c

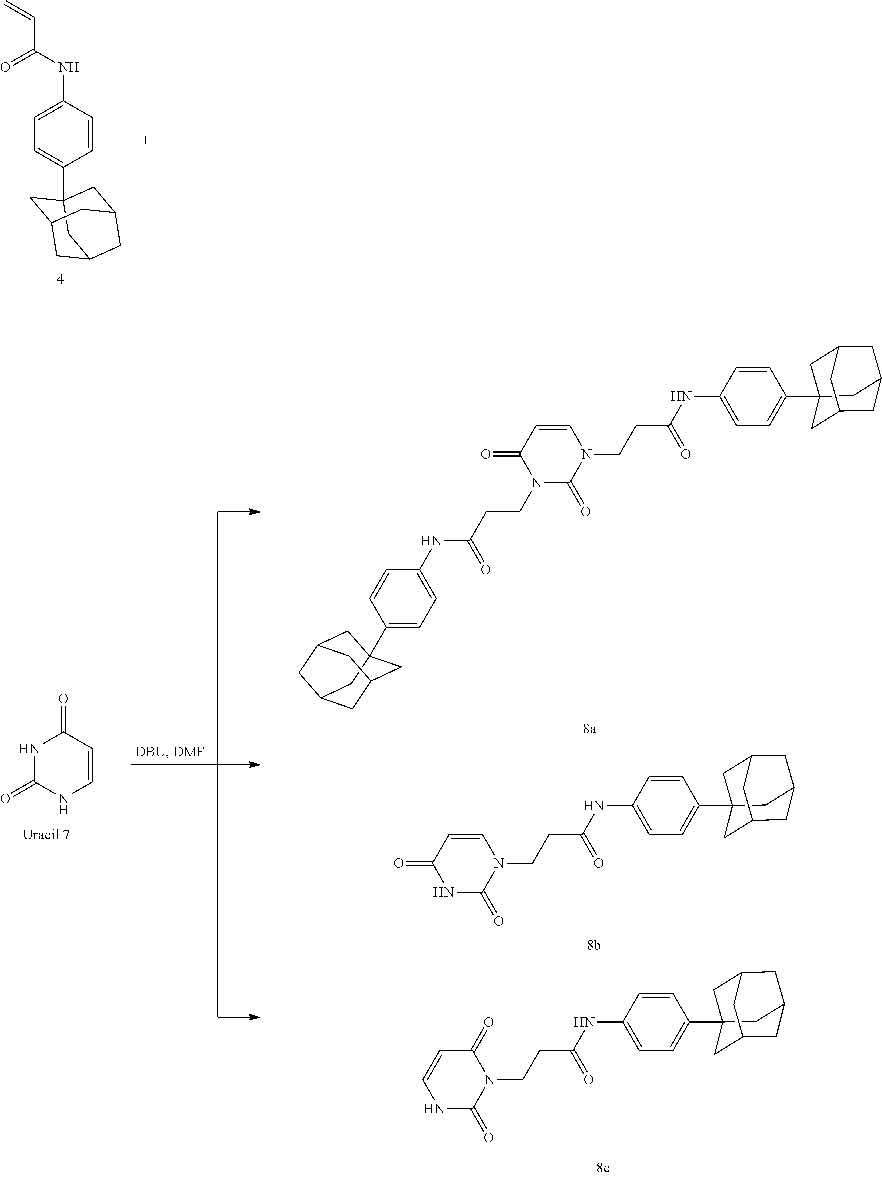 [SMILES: C=CC(=O)Nc1ccc(C23CC4CC(CC(C4)C2)C3)cc1 ; O=c1cc[nH]c(=O)[nH]1 ; O=C(CCn1ccc(=O)n(CCC(=O)Nc2ccc(C34CC5CC(CC(C5)C3)C4)cc2)c1=O)Nc1ccc(C23CC4CC(CC(C4)C2)C3)cc1 ; O=C(CCn1ccc(=O)[nH]c1=O)Nc1ccc(C23CC4CC(CC(C4)C2)C3)cc1 ; O=C(CCn1c(=O)cc[nH]c1=O)Nc1ccc(C23CC4CC(CC(C4)C2)C3)cc1]

Equal amounts of uracil 7 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compounds 8a, 8b, and 8c in sequence, with a total yield of 85% to 95%.

Analytic data of compound 8a: ¹H NMR (DMSO-d₆, 600 MHz) δ 9.92 (s, 1H), 9.87 (s, 1H), 7.66 (d, J=7.9 Hz, 1H), 7.48-7.45 (m, 4H), 7.28-7.21 (m, 4H), 5.66 (d, J=7.9 Hz, 1H), 4.08 (t, J=7.5 Hz, 2H), 3.99 (t, J=6.6 Hz, 2H), 2.70 (t, J=6.6 Hz, 2H), 2.54 (t, J=7.6 Hz, 2H), 2.05-2.00 (m, 6H), 1.81 (d, J=3.1 Hz, 12H), 1.74-1.67 (m, 12H). LRMS calcd for C₄₂H₅₀N₄O₄ [M+Na]⁺ 697.37, found 697.37.

Analytic data of compound 8b: ¹H NMR (DMSO-d₆, 600 MHz) o 11.25 (s, 1H), 9.94 (s, 1H), 7.58 (d, J=7.9 Hz, 1H), 7.47-7.44 (m, 2H), 7.28-7.23 (m, 2H), 5.50 (d, J=7.9 Hz, 1H), 3.93 (t, J=6.5 Hz, 2H), 2.68 (t, J=6.6 Hz, 2H), 2.03 (p, J=3.3 Hz, 3H), 1.81 (d, J=3.3 Hz, 6H), 1.74-1.68 (m, 6H). LRMS calcd for C₂₃H₂₇N₃O₃ [M+Na]⁺ 416.20, found 416.19.

Analytic data of compound 8c: ¹H NMR (DMSO-d₆, 600 MHz) δ 11.12 (d, J=5.3 Hz, 1H), 9.87 (s, 1H), 7.48-7.45 (m, 2H), 7.44-7.41 (m, 1H), 7.27-7.23 (m, 2H), 5.59 (d, J=7.5 Hz, 1H), 4.08-4.00 (m, 2H), 2.57-2.52 (m, 2H), 2.04 (p, J=3.3 Hz, 3H), 1.83 (d, J=3.3 Hz, 6H), 1.72 (q, J=3.8 Hz, 6H). LRMS calcd for C₂₃H₂₇N₃O₃ [M+Na]⁺ 416.20, found 416.20.

### Example 3: Preparation of Compounds 10a, 10b, and 10c

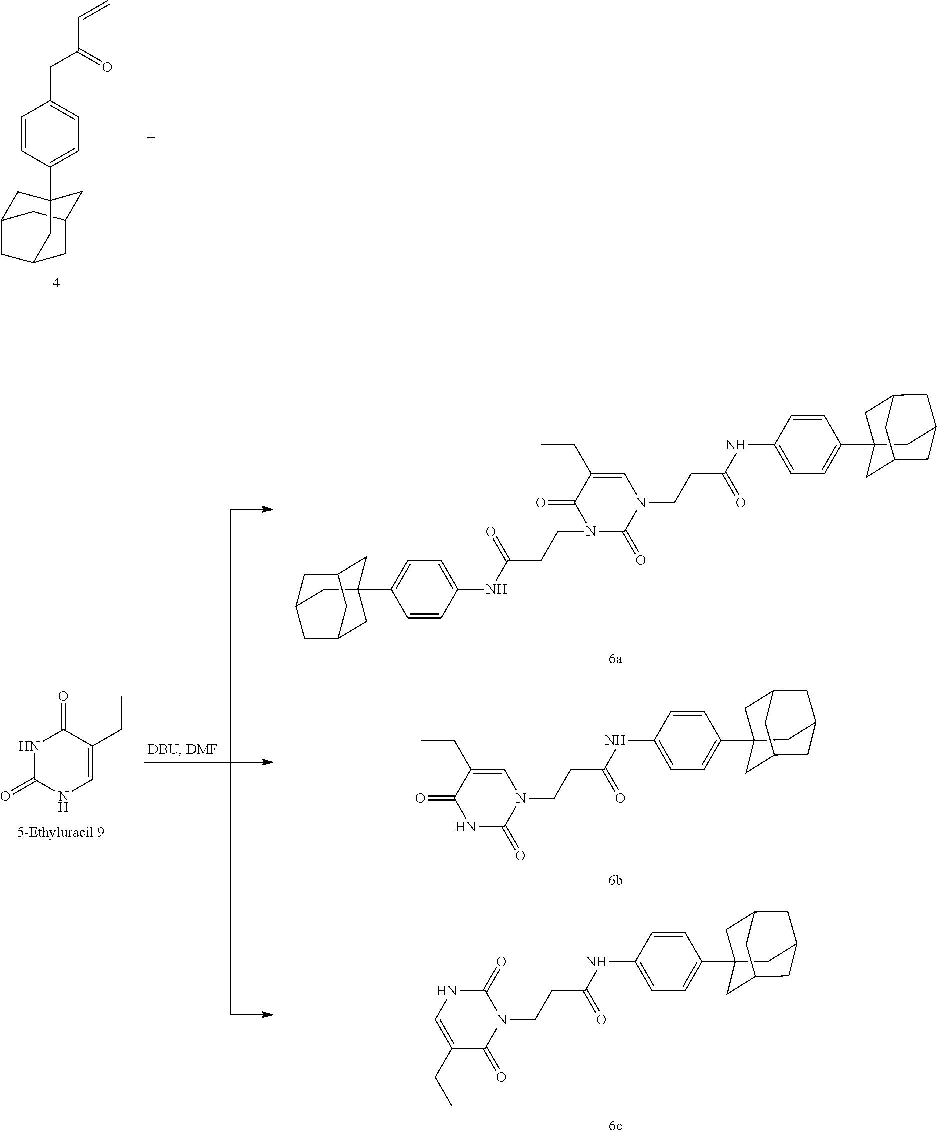

Equal amounts of 5-ethyluracil 9 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compounds 10a, 10b, and 10c in sequence, with a total yield of 70% to 75%.

Analytic data of compound 10a: ¹H NMR (600 MHz, DMSO-d6) δ 9.88 (s, 1H), 9.80 (s, 1H), 7.57-7.51 (m, 4H), 7.41 (d, J=1.3 Hz, 1H), 7.32-7.26 (m, 4H), 4.03 (td, J=9.0, 7.0 Hz, 4H), 2.83-2.71 (m, 4H), 2.37 (q, J=5.9 Hz, 2H), 2.11 (m, 6H), 1.92 (d, J=4.0 Hz, 12H), 1.77 (t, J=4.1 Hz, 12H), 1.08 (t, J=s, 3H). LRMS calcd for C₄₄H₅₄N₄O₄ [M+H]⁺ 703.42, found 703.43.

Analytic data of compound 10b: ¹H NMR (600 MHz, DMSO-d₆) δ 11.32 (s, 1H), 9.95 (s, 1H), 7.48-7.45 (m, 2H), 7.42 (s, 1H), 7.23-7.18 (m, 3H), 4.09 (t, J=7.6 Hz, 1H), 3.95 (t, J=6.7 Hz, 1H), 2.69 (t, J=6.8 Hz, 1H), 2.47 (t, J=7.8 Hz, 1H), 2.08 (s, 3H), 1.87 (t, J=3.7 Hz, 6H), 1.75 (s, 6H), 1.03-1.01 (m, 1H), 0.98 (td, J=7.5, 1.6 Hz, 2H). LRMS calcd for C₂₅H₃₁N₃O₃ [M+H]⁺ 422.24, found 422.38.

Analytic data of compound 10c: ¹H NMR (600 MHz, DMSO-d₆) δ 11.23 (s, 1H), 9.93 (s, 1H), 7.49-7.44 (m, 2H), 7.40 (s, 1H), 7.28-7.22 (m, 3H), 4.07 (t, J=7.6 Hz, 1H), 3.93 (t, J=6.7 Hz, 1H), 2.67 (t, J=6.8 Hz, 1H), 2.55 (t, J=7.8 Hz, 1H), 2.05 (s, 3H), 1.83 (t, J=3.7 Hz, 6H), 1.73 (s, 6H), 1.05-1.00 (m, 1H), 0.96 (td, J=7.5, 1.6 Hz, 2H). LRMS calcd for C25H31N303 [M +H]⁺ 422.24, found 422.35.

### Example 4: Preparation of Compounds 12a, 12b, and 12c

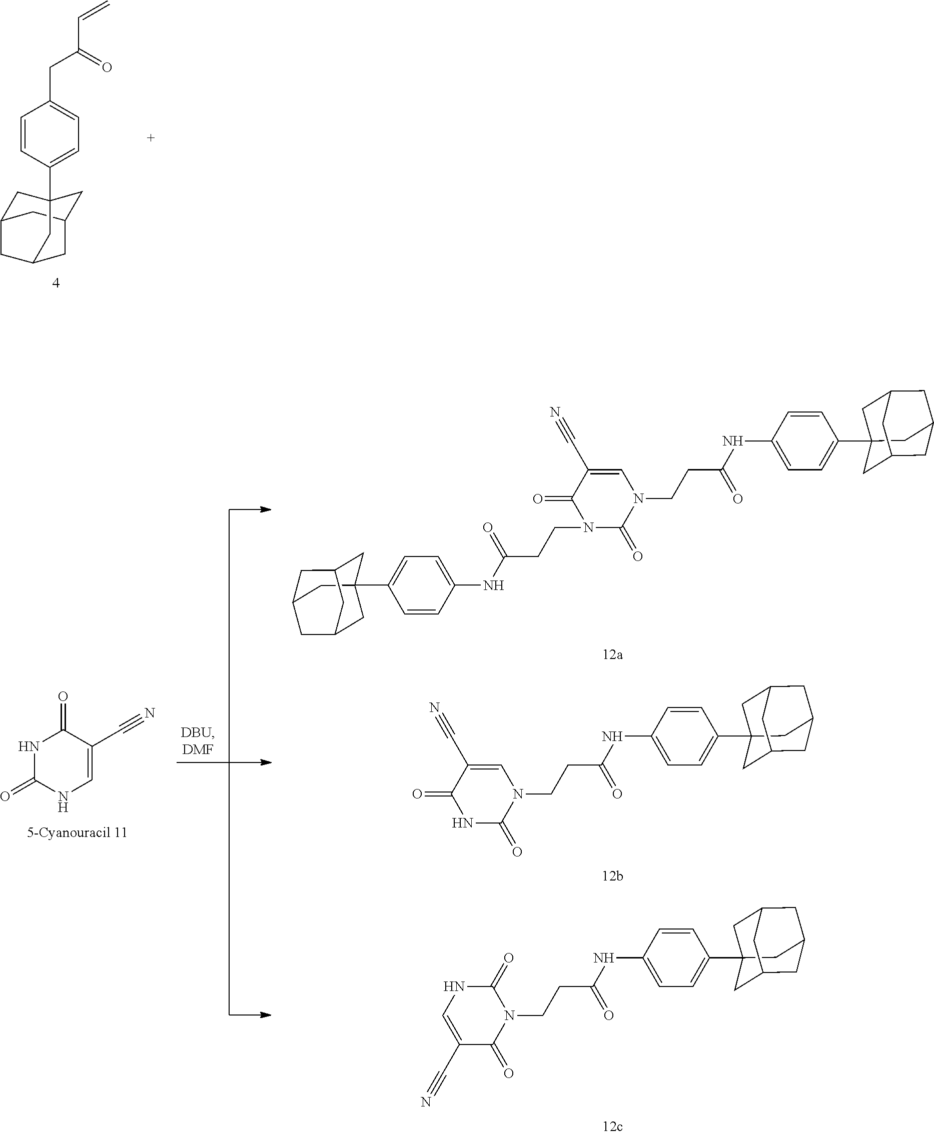 [SMILES: C=CC(=O)Cc1ccc(C23CC4CC(CC(C4)C2)C3)cc1 ; N#Cc1cn(CCC(=O)Nc2ccc(C34CC5CC(CC(C5)C3)C4)cc2)c(=O)n(CCC(=O)Nc2ccc(C34CC5CC(CC(C5)C3)C4)cc2)c1=O ; N#Cc1c[nH]c(=O)[nH]c1=O ; N#Cc1cn(CCC(=O)Nc2ccc(C34CC5CC(CC(C5)C3)C4)cc2)c(=O)[nH]c1=O ; N#Cc1c[nH]c(=O)n(CCC(=O)Nc2ccc(C34CC5CC(CC(C5)C3)C4)cc2)c1=O]

Equal amounts of 5-cyanouracil 11 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compounds 12a, 12b, and 12c, with a total yield of 70% to 80%.

Analytic data of compound 12a: ¹H NMR (600 MHz, DMSO-d₆) δ 9.84 (s, 1H), 9.82 (s, 1H), 7.62 (d, J=1.3 Hz H), 7.57-7.51 (m, 4H), 7.32-7.26 (m, 4H), 4.11-4.02 (m, 4H), 2.83-2.70 (m, 4H), 2.11-2.08 (m, 6H), 1.92 (d, J=3.8 Hz, 12H), 1.77-1.70 (m, 12H). LRMS calcd for C₄₃H₄₉N₅O₄ [M+H]⁺700.39, found 700.37.

Analytic data of compound 12b: ¹H NMR (600 MHz, DMSO-d₆) δ 9.90 (s, 1H), 8.49 (s, 1H), 7.56-7.37 (m, 2H), 7.33-7.16 (m, 2H), 4.05 (t, J=7.4 Hz, 2H), 2.56 (t, J=7.4 Hz, 2H), 2.04 (p, J=3.3 Hz, 3H), 1.83 (d, J=3.0 Hz, 6H), 1.73-1.71 (m, 6H). LRMS calcd for C₂₄H₂₆N₄O₃ [M+H]⁺419.21, found 419.24.

Analytic data of compound 12c: ¹H NMR (600 MHz, DMSO-d₆) δ 9.89 (s, 1H), 8.51 (s, 1H), 7.54-7.39 (m, 2H), 7.35-7.12 (m, 2H), 4.08 (t, J=6.9 Hz, 2H), 2.58 (t, J=7.4 Hz, 2H), 2.08 (p, J=3.3 Hz, 3H), 1.89 (d, J=3.0 Hz, 6H), 1.75-1.73 (m, 6H). LRMS calcd for C₂₄H₂₆N₄O₃ [M+H]⁺ 419.21, found 419.22.

### Example 5: Preparation of Compounds 14a, 14b, and 14c

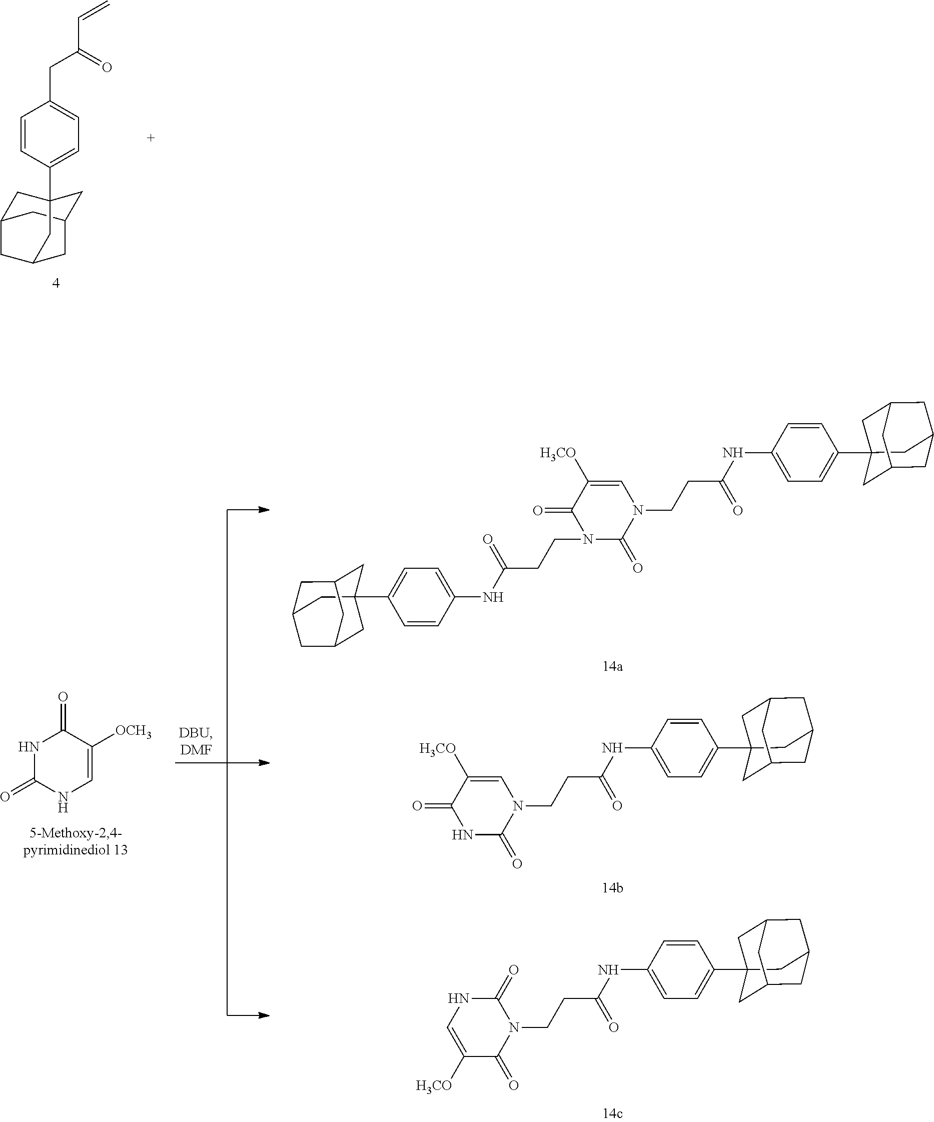 [SMILES: C=CC(=O)Cc1ccc(C23CC4CC(CC(C4)C2)C3)cc1 ; COc1cn(CCC(=O)Nc2ccc(C34CC5CC(CC(C5)C3)C4)cc2)c(=O)n(CCC(=O)Nc2ccc(C34CC5CC(CC(C5)C3)C4)cc2)c1=O ; COc1c[nH]c(=O)[nH]c1=O ; COc1cn(CCC(=O)Nc2ccc(C34CC5CC(CC(C5)C3)C4)cc2)c(=O)[nH]c1=O ; COc1c[nH]c(=O)n(CCC(=O)Nc2ccc(C34CC5CC(CC(C5)C3)C4)cc2)c1=O]

Equal amounts of 5-methoxyuracil 13 and 1,8-diazabicyclo [5.4.0]undec-7-ene, together with excess compound 4, were dissolved in N,N-dimethylformamide (DMF), and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compounds 14a, 14b, and 14c, with a total yield of 65% to 75%.

Analytic data of compound 14a: ¹H NMR (600 MHz, DMSO-d₆) δ 11.40 (s, 2H), 7.55-7.47 (m, 4H), 7.32 (s, 1H), 7.30-7.26 (m, 4H), 3.94 (t, J=6.8 Hz, 4H), 3.58 (s, 3H), 2.70 (t, J=6.0 Hz, 4H), 2.10-1.96 (m, 6H), 1.85 (d, J=3.8 Hz, 12H), 1.79-1.67 (m, 12H). LRMS calcd for C₄₃H₅₂N₄O₅ [M+H]⁺705.40, found 705.41.

Analytic data of compound 14b: ¹H NMR (600 MHz, DMSO-d₆) δ 10.77 (d, J=6.0 Hz, 1H), 9.89 (s, 1H), 7.51-7.41 (m, 2H), 7.31-7.21 (m, 2H), 7.14-6.98 (m, 1H), 4.06 (t, J=7.6 Hz, 2H), 3.61 (d, J=1.7 Hz, 3H), 2.55 (t, J=7.6 Hz, 2H), 2.05 (s, 3H), 1.83 (d, J=3.0 Hz, 6H), 1.73 (d, J=4.3 Hz, 6H). LRMS calcd for C₂₄H₂₉N₃O₄ [M+H]⁺424.22, found 424.34.

Analytic data of compound 14a: ¹H NMR (600 MHz, DMSO-d₆) δ 11.43 (s, 1H), 9.95 (s, 1H), 7.56-7.44 (m, 2H), 7.29 (s, 1H), 7.29-7.24 (m, 2H), 3.92 (t, J=6.8 Hz, 2H), 3.55 (s, 3H), 2.68 (t, J=6.8 Hz, 2H), 2.09-1.99 (m, 3H), 1.83 (d, J=3.0 Hz, 6H), 1.78-1.66 (m, 6H). LRMS calcd for C₂₄H₂₉N₃O₄ [M+H]⁺424.22, found 424.36.

### Example 6: Preparation of Compounds 16a, 16b, and 16c

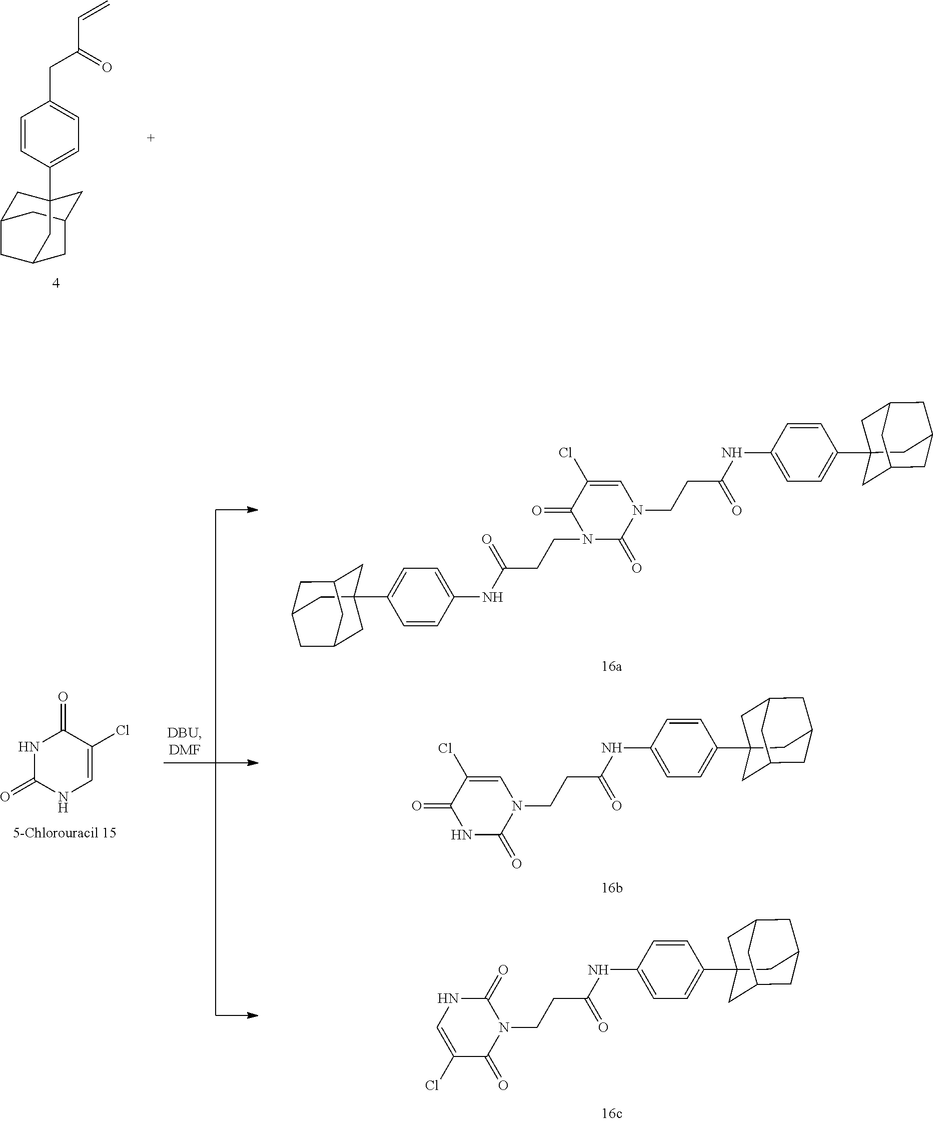 [SMILES: C=CC(=O)Cc1ccc(C23CC4CC(CC(C4)C2)C3)cc1 ; O=C(CCn1cc(Cl)c(=O)n(CCC(=O)Nc2ccc(C34CC5CC(CC(C5)C3)C4)cc2)c1=O)Nc1ccc(C23CC4CC(CC(C4)C2)C3)cc1 ; O=c1[nH]cc(Cl)c(=O)[nH]1 ; O=C(CCn1cc(Cl)c(=O)[nH]c1=O)Nc1ccc(C23CC4CC(CC(C4)C2)C3)cc1 ; O=C(CCn1c(=O)[nH]cc(Cl)c1=O)Nc1ccc(C23CC4CC(CC(C4)C2)C3)cc1]

Equal amounts of 5-chlorouracil 15 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compounds 16a, 16b, and 16c, with a total yield of 70% to 75%.

Analytic data of compound 16a: ¹H NMR (600 MHz, DMSO-d₆) δ 11.48 (s, 2H), 7.92 (s, 1H), 7.50 (d, J=6.8 Hz, 4H), 7.24 (d, J=8.8 Hz, 4H), 4.12 (t, J=8.5 Hz, 4H), 2.59 (t, J=5.5 Hz, 4H), 2.07-2.03 (m, 6H), 1.84 (d, J=4.0 Hz, 12H), 1.75-1.66 (m,12H). LRMS calcd for C₄₂H₄₉ClN₄O₄ [M+H]⁺ 709.35, found 709.37.

Analytic data of compound 16b: ¹H NMR (600 MHz, DMSO-d₆) δ 11.56 (s, 1H), 9.91 (s, 1H), 7.92 (s, 1H), 7.47 (d, J=8.6 Hz, 2H), 7.26 (d, J=8.6 Hz, 2H), 4.09 (t, J=7.5 Hz, 2H), 2.56 (t, J=7.5 Hz, 2H), 2.06-2.02 (m, 3H), 1.83 (d, J=3.0 Hz, 6H), 1.73 (d, J=3.8 Hz, 6H). LRMS calcd for C₂₃H₂₆ClN₃O₃ [M+H]⁺428.17, found 428.16.

Analytic data of compound 16c: ¹H NMR (600 MHz, DMSO-d₆) δ 11.52 (s, 1H), 9.94 (s, 1H), 7.94 (s, 1H), 7.50 (d, J=7.2 Hz, 2H), 7.29 (d, J=8.0 Hz, 2H), 4.05 (t, J=7.1 Hz, 2H), 2.59 (t, J=6.5 Hz, 2H), 2.09-2.01 (m, 3H), 1.85 (d, J=2.8 Hz, 6H), 1.75 (d, J=3.9 Hz, 6H). LRMS calcd for C₂₃H₂₆ClN₃O₃ [M+H]⁺428.17, found 428.19.

### Example 7: Preparation of Compounds 18a, 18b, and 18c

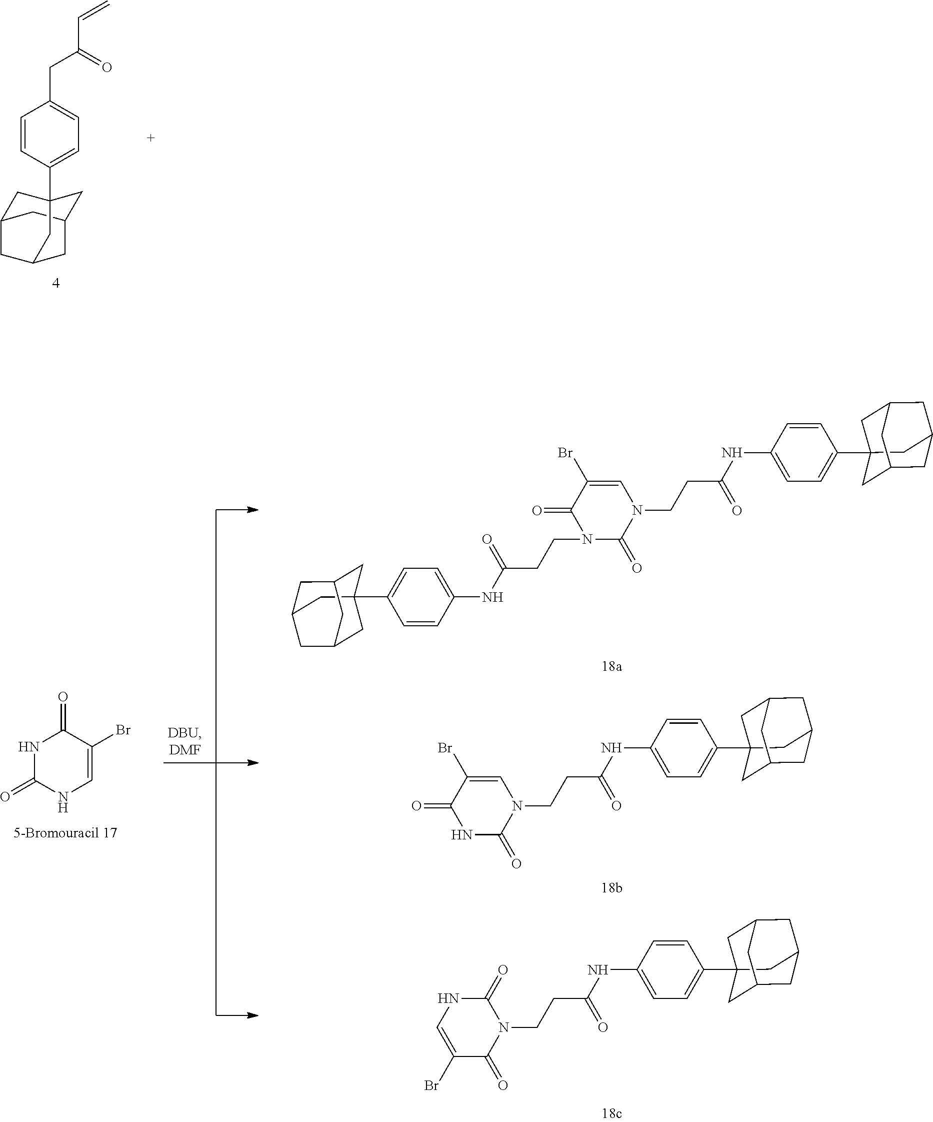 [SMILES: C=CC(=O)Cc1ccc(C23CC4CC(CC(C4)C2)C3)cc1 ; O=C(CCn1cc(Br)c(=O)n(CCC(=O)Nc2ccc(C34CC5CC(CC(C5)C3)C4)cc2)c1=O)Nc1ccc(C23CC4CC(CC(C4)C2)C3)cc1 ; O=c1[nH]cc(Br)c(=O)[nH]1 ; O=C(CCn1cc(Br)c(=O)[nH]c1=O)Nc1ccc(C23CC4CC(CC(C4)C2)C3)cc1 ; O=C(CCn1c(=O)[nH]cc(Br)c1=O)Nc1ccc(C23CC4CC(CC(C4)C2)C3)cc1]

Equal amounts of 5-bromouracil 17 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compounds 18a, 18b, and 18c, with a total yield of 70% to 80%.

Analytic data of compound 18a: ¹H NMR (600 MHz, DMSO-d₆) δ 11.50 (s, 2H), 7.98 (s, 1H), 7.49 (d, J=2.8 Hz, 4H), 7.48 (s, 4H), 4.11 (t, J=5.2 Hz, 4H), 2.58 (t, J=5.8 Hz, 4H), 2.04 (s, 6H), 1.84 (d, J=3.2 Hz, 12H), 1.75 (s, 12H). LRMS calcd for C₂₃H₂₆BrN₃O₃ [M+H]⁺ 753.30, found 753.32.

Analytic data of compound 18b: ¹H NMR (600 MHz, DMSO-d₆) δ 11.60 (s, 1H), 9.93 (s, 1H), 7.99 (s, 1H), 7.47 (d, J=2.1 Hz, 2H), 7.45 (s, 2H), 4.09 (t, J=7.4 Hz, 2H), 2.56 (t, J=7.5 Hz, 2H), 2.04 (s, 3H), 1.83 (d, J=2.9 Hz, 6H), 1.72 (s, 6H). LRMS calcd for C₂₃H₂₆BrN₃O₃ [M+H]⁺472.12, found 472.16.

Analytic data of compound 18c: ¹H NMR (600 MHz, DMSO-d₆) δ 11.58 (s, 1H), 9.91 (s, 1H), 7.97 (s, 1H), 7.48 (d, J=2.4Hz, 2H), 7.44 (s, 2H), 4.07 (t, J=6.3 Hz, 2H), 2.59 (t, J=6.0 Hz, 2H), 2.06 (s, 3H), 1.86 (d, J=2.0 Hz, 6H), 1.75 (s, 6H). LRMS calcd C₂₃H₂₆BrN₃O₃ [M+H]⁺ 472.12, found 472.14.

### Example 8: Preparation of Compounds 20a, 20b, and 20c

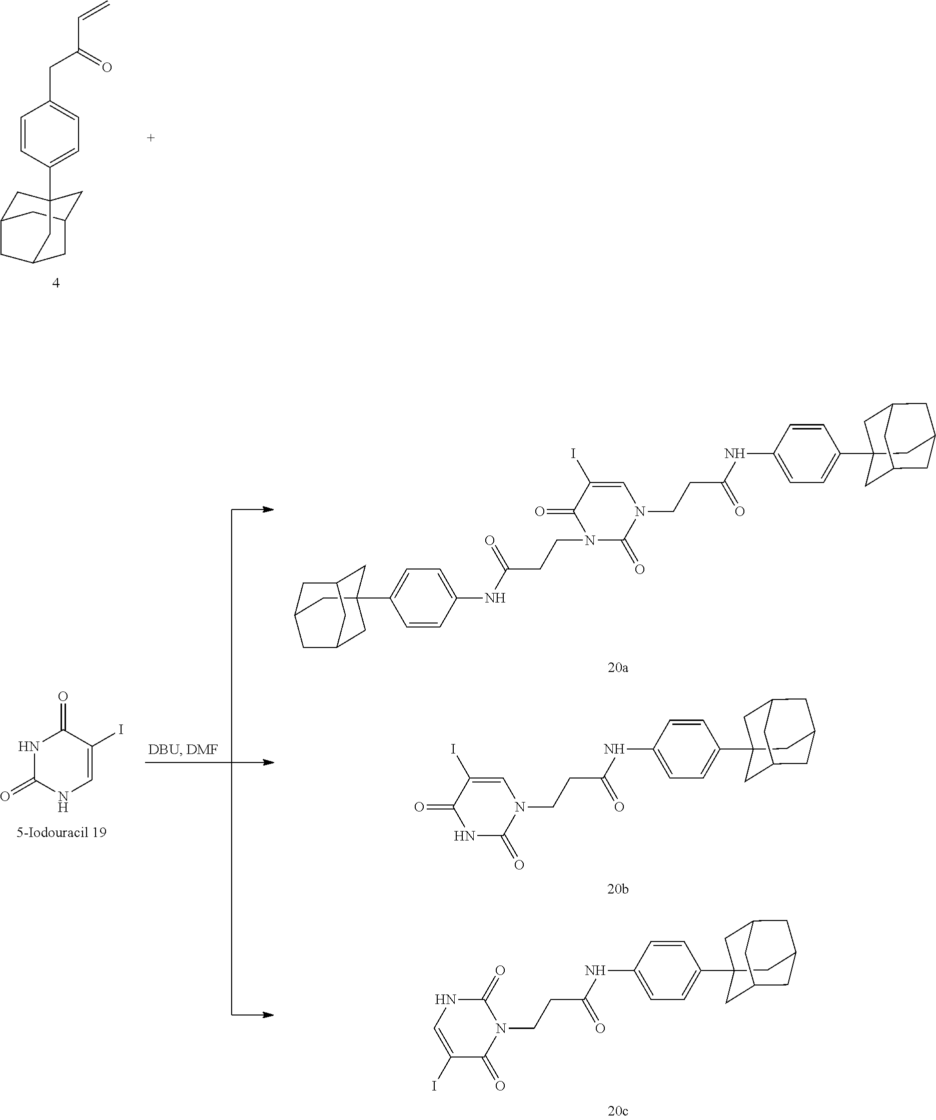 [SMILES: C=CC(=O)Cc1ccc(C23CC4CC(CC(C4)C2)C3)cc1 ; O=C(CCn1cc(I)c(=O)n(CCC(=O)Nc2ccc(C34CC5CC(CC(C5)C3)C4)cc2)c1=O)Nc1ccc(C23CC4CC(CC(C4)C2)C3)cc1 ; O=c1[nH]cc(I)c(=O)[nH]1 ; O=C(CCn1cc(I)c(=O)[nH]c1=O)Nc1ccc(C23CC4CC(CC(C4)C2)C3)cc1 ; O=C(CCn1c(=O)[nH]cc(I)c1=O)Nc1ccc(C23CC4CC(CC(C4)C2)C3)cc1]

Equal amounts of 5-iodouracil 19 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compounds 20a, 20b, and 20c, with a total yield of 70% to 75%.

Analytic data of compound 20a: ¹H NMR (600 MHz, DMSO-d₆) δ 9.90 (d, J=39.5 Hz, 2H), 7.66 (d, J=7.9 Hz, 1H), 7.51-7.43 (m, 4H), 7.28-7.22 (m, 4H), 4.09 (t, J=7.6 Hz, 2H), 4.00 (t, J=6.6 Hz, 2H), 2.71 (t, J=6.6 Hz, 2H), 2.55 (t, J=7.7 Hz, 2H), 2.03 (d, J=4.3 Hz, 6H), 1.85-1.79 (m, 12H), 1.71 (q, J=11.8 Hz, 12H). LRMS calcd for C₄₂H₄₉IN₄O₄ [M+H]⁺801.29, found 801.28.

Analytic data of compound 20b: ¹H NMR (600 MHz, DMSO-d₆) δ 11.25 (d, J=2.3 Hz, 1H), 9.94 (s, 1H), 7.58 (d, J=7.8 Hz, 1H), 7.49-7.43 (m, 2H), 7.29-7.21 (m, 2H), 3.93 (t, J=6.5 Hz, 2H), 2.69 (t, J=6.6 Hz, 2H), 2.05-2.00 (m, 3H), 1.82 (d, J=3.1 Hz, 6H), 1.74-1.69 (m, 6H). LRMS calcd for C₂₃H₂₆IN₃O₃ [M+Na]⁺542.09, found 542.06.

Analytic data of compound 20c: ¹H NMR (600 MHz, DMSO-d₆) δ 11.12 (d, J=5.4 Hz, 1H), 9.87 (s, 1H), 7.48-7.44 (m, 2H), 7.42 (dd, J=7.5, 5.8 Hz, 1H), 7.25 (d, J=8.6 Hz, 2H), 4.08-3.98 (m, 2H), 2.57-2.51 (m, 2H), 2.03 (s, 3H), 1.82 (d, J=2.2 Hz, 6H), 1.74-1.68 (m, 6H). LRMS calcd for C₂₃H₂₆IN₃O₃ [M+Na]⁺542.09, found 542.07.

### Example 9: Preparation of Compound 22

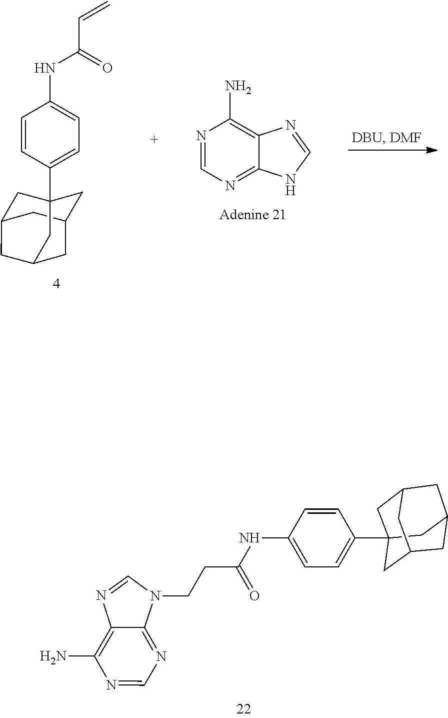 [SMILES: C=CC(=O)Nc1ccc(C23CC4CC(CC(C4)C2)C3)cc1 ; Nc1ncnc2[nH]cnc12 ; Nc1ncnc2c1ncn2CCC(=O)Nc1ccc(C23CC4CC(CC(C4)C2)C3)cc1]

Equal amounts of adenine 21 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compound 22, with a total yield of 65% to 70%.

Analytic data of compound 22: ¹H NMR (DMSO-d₆, 600 MHz) δ 9.91 (s, 1H), 8.16 (s, 1H), 8.05 (s, 1H), 7.48-7.22 (m, 2H), 7.17 (s, 2H), 4.43 (t, J=6.7 Hz, 2H), 2.92 (t, J=6.7 Hz, 2H), 2.03 (q, J=3.2 Hz, 3H), 1.82 (d, J=3.0 Hz, 6H), 1.73-1.69 (m, 6H). LRMS calcd for C₂₄H₂₈N₆O [M+H]⁺417.24, found 417.27.

### Example 10: Preparation of Compound 24

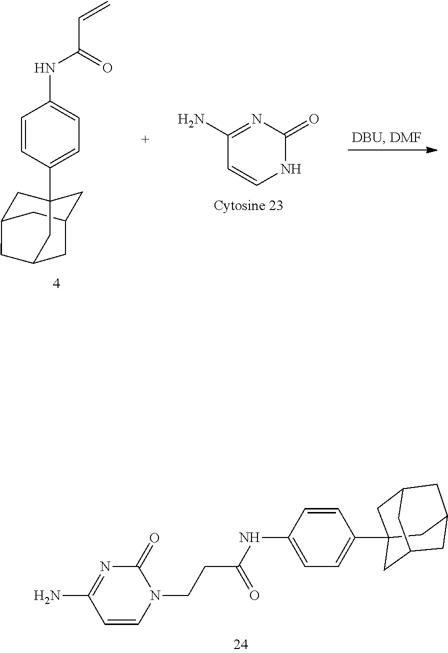 [SMILES: C=CC(=O)Nc1ccc(C23CC4CC(CC(C4)C2)C3)cc1 ; Nc1cc[nH]c(=O)n1 ; Nc1ccn(CCC(=O)Nc2ccc(C34CC5CC(CC(C5)C3)C4)cc2)c(=O)n1]

Equal amounts of cytosine 23 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compound 24, with a total yield of 70% to 75%.

Analytic data of compound 24: ¹H NMR (600 MHz, DMSO-d₆) δ 9.90 (d, J=19.1 Hz, 1H), 7.48 (d, J=8.4 Hz, 2H), 7.25 (dd, J=8.8, 3.2 Hz, 2H), 4.10 (t, J=7.5 Hz, 1H), 4.01 (t, J=6.5 Hz, 1H), 2.72 (t, J=6.5 Hz, 1H), 2.56 (t, J=7.6 Hz, 1H), 2.03 (s, 3H), 1.81 (d, J=3.0 Hz, 6H), 1.71 (d, J=3.7 Hz, 6H). LRMS calcd for C₂₃H₂₈N₄O₂ [M+Na]⁺ 415.21, found 415.20.

### Example 11: Preparation of Compound 26

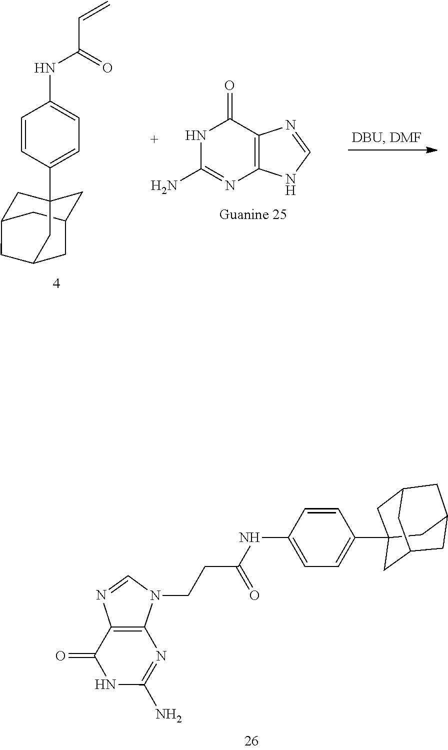 [SMILES: C=CC(=O)Nc1ccc(C23CC4CC(CC(C4)C2)C3)cc1 ; Nc1nc2[nH]cnc2c(=O)[nH]1 ; Nc1nc2c(ncn2CCC(=O)Nc2ccc(C34CC5CC(CC(C5)C3)C4)cc2)c(=O)[nH]1]

Equal amounts of guanine 25 and 1,8-diazabicyclo [5.4.0]undec-7-ene, together with excess compound 4, were dissolved in N,N-dimethylformamide, and then allowed to react under refluxing. The reaction was monitored by TLC until completion, and then quenched with water. The aqueous phase was extracted three times with dichloromethane. The organic phases were combined, and dried over anhydrous Na₂SO₄. The organic solvent was removed by rotatory evaporation under reduced pressure. The residue was isolated and purified by flash column chromatography, to obtain compound 26, with a total yield of 65% to 70%.

Analytic data of compound 26: ¹H NMR (600 MHz, DMSO-d₆) δ 9.62 (d, J=1.1 Hz, 2H), 7.87 (s, 1H), 7.56-7.49 (m, 2H), 7.30-7.24 (m, 2H), 6.04 (d, J=7.1 Hz, 1H), 5.48 (d, J=7.1 Hz, 1H), 4.39 (t, J=9.9 Hz, 2H), 2.88-2.80 (m, 2H), 2.18-2.04 (m, 3H), 1.93 (d, J=8.0 Hz, 6H), 1.78 (t, J=8.1 Hz, 6H). LRMS calcd for C₂₄H₂₈N₆O₂ [M+H]⁺ 433.24, found 433.26.

The beneficial effects of the present invention were demonstrated by the following experimental examples.

### Experimental Example 1: Inhibition of Compounds on Proliferation of SCC Cells

### 1. Experimental Methods

Squamous cell carcinoma cell lines: HSC-3, HSC-4.

The above two tumor cell lines were cultured in a standardized incubator at 37° C. under 5% CO₂; the cells in logarithmic growth phase were digested and counted, followed by seeding in a 96-well plate at 3000 cells/well, and then cultured overnight in an incubator. The stock solution of each compound was prepared using DMSO at a concentration of 10 mM, and diluted sequentially (0, 1.25, 2.5, 5, 10 μM) before treating the cells. The control group was added with the highest concentration of DMSO, and then cultivated continuously in an incubator for 48 h. The CCK8 cell proliferation kit was used to detect the effect of each compound on the proliferation of tumor cells. Moreover, the experimental results of representative compounds were analyzed, and their IC₅₀ values were calculated to evaluate the effect of the compounds on the proliferation of tumor cells.

### 2. Experimental Results

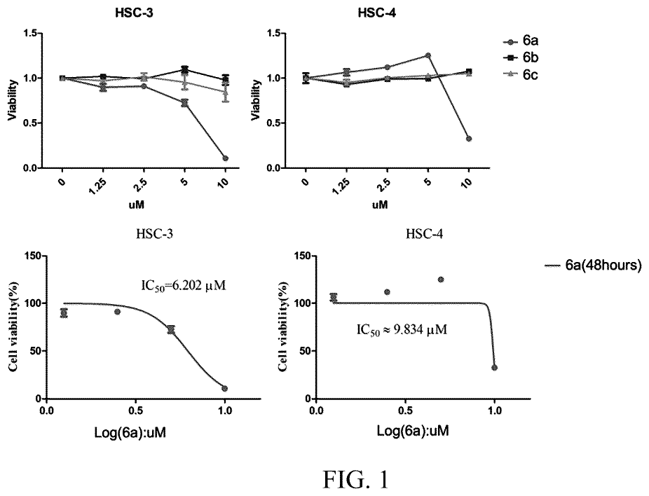

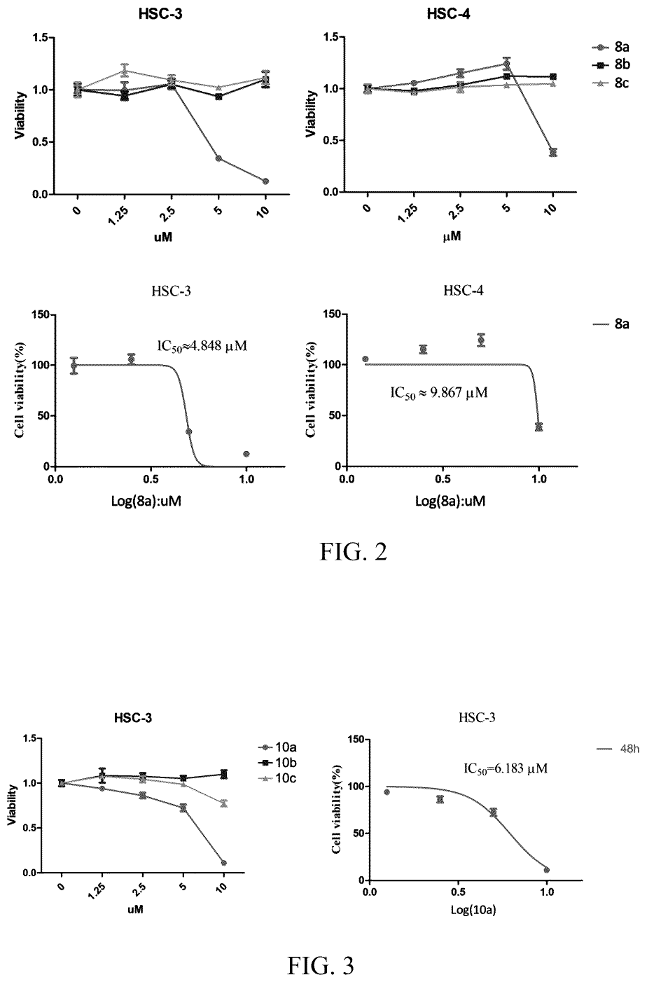

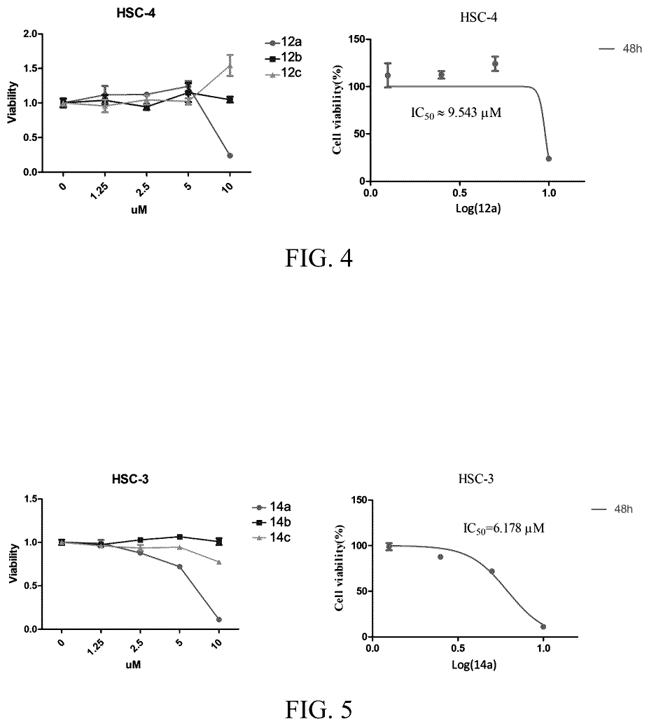

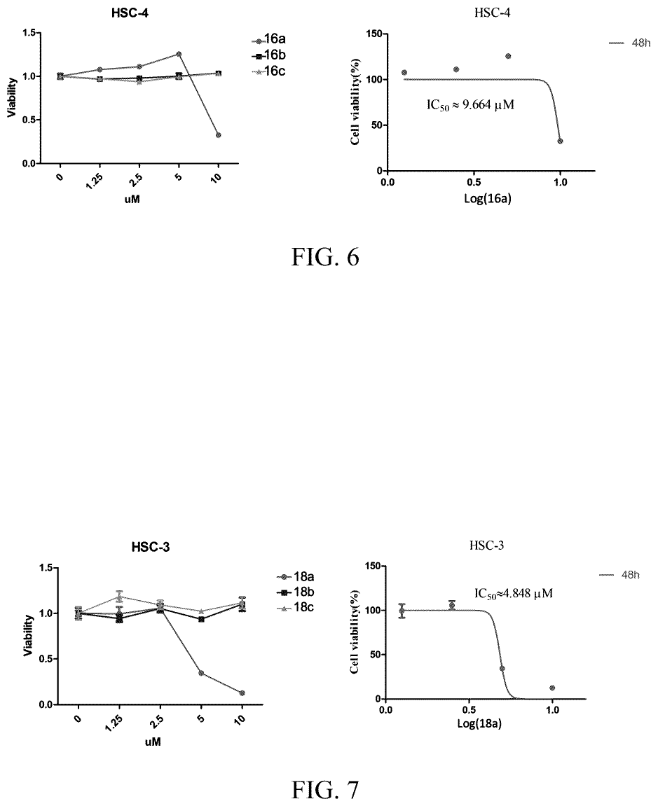

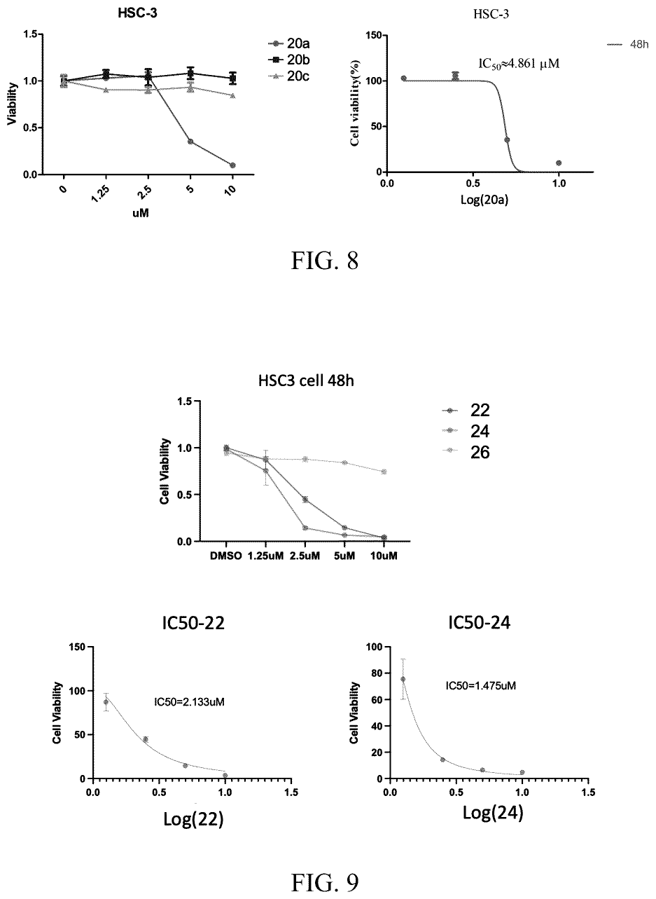

As shown in FIGS. 1-9, the compounds of the present invention have a significant inhibitory effect on the proliferation of SCC cells. Among them, the IC₅₀ values of compound 6a against two SCC cells were 6.202 μM and 9.834 μM, respectively (FIG. 1); the IC₅₀ values of compound 8a against two SCC cells were 4.848 μM and 9.868 μM, respectively (FIG. 2); the IC₅₀ values of compound 10a against SCC cells was 6.183 μM (FIG. 3); the IC₅₀ value of compound 12a against SCC cells was 9.543 μM (FIG. 4); the IC₅₀ value of compound 14a against SCC cells was 6.178 μM (FIG. 5); the IC₅₀ value of compound 16a against SCC cells was 9.664 μM (FIG. 6); the ICs0 value of compound 18a against SCC cells was 4.848 μM (FIG. 7); the IC₅₀ value of compound 20a against SCC cells was 4.861 μM (FIG. 8); the IC₅₀ values of compounds 22 and 24 against SCC cells were 2.133 μM and 1.475 μM, respectively (FIG. 9).

The above experimental results indicated that the compounds provided in the present invention could effectively inhibit the proliferation of SCC cells, and could be used in the manufacture of medicaments for treating SCC.

1. Compounds represented by formula I, pharmaceutically acceptable salts thereof, and stereoisomers thereof: 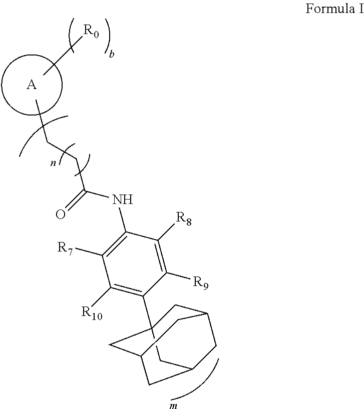
wherein, ring A is selected from N-containing fused rings or 5-6-membered unsaturated N-containing heterocycles;
b is an integer selected from 0 to 6;
R₀ is each independently selected from the group consisting of ═O, ═S, H, amino, halogen, C₁₋₆ alkyl, C₁₋₆ alkoxy, hydroxyl, carboxyl, cyano, alkylamine, heterocycle-substituted alkyl, (aromatic ring)-substituted alkyl;
m is an integer selected from 1 to 3; n is an integer selected from 0 to 5; R₇, R8, R9, and R10 are each independently selected from the group consisting of H, amino, halogen, C₁₋₆ alkyl, C₁₋₆ alkoxy, halogen, hydroxyl, and carboxyl.

2. The compound according to claim 1, pharmaceutically acceptable salts thereof, and stereoisomers thereof, characterized in that the structure of the compound is as represented by formula II: 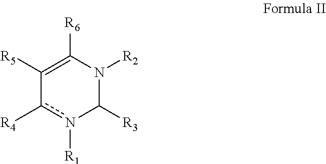
wherein, [symbol] represents [symbol] or [symbol] ;
R₁ is selected from the group consisting of absence, H or 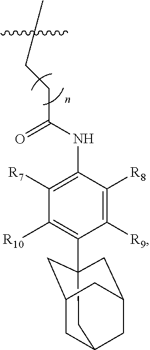
R₂ is selected from the group consisting of H or 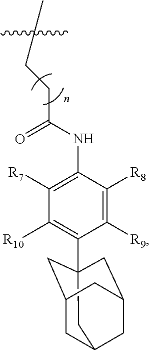
and provided that R₁ is absence or H, R₂ is not H;
n is an integer selected from 0 to 3; R₇, R₈, R₉, and R₁₀ are each independently selected from the group consisting of H, amino, halogen, C₁₋₄ alkyl, C₁₋₄ alkoxy, halogen, hydroxyl, and carboxyl;
R₃ is selected from the group consisting of ═O, ═S, H, amino, halogen, C₁₋₄ alkyl, C₁₋₄ alkoxy, hydroxyl, and carboxyl;
R₄ is selected from the group consisting of ═O, ═S, H, amino, halogen, C₁₋₄ alkyl, C₁₋₄ alkoxy, hydroxyl, and carboxyl;
and at least one of R₃ and R₄ is ═O or —S;
R₅ is selected from the group consisting of H, amino, halogen, C₁₋₄ alkyl, C₁₋₄ alkoxy, hydroxyl, carboxyl, cyano, alkylamine, heterocycle-substituted alkyl, and (aromatic ring)-substituted alkyl;
R₆ is selected from the group consisting of H, amino, halogen, C₁₋₄ alkyl, C₁₋₄ alkoxy, hydroxyl, and carboxyl.

3. The compound according to claim 2, pharmaceutically acceptable salts thereof, and stereoisomers thereof, characterized in that the structure of the compound is as represented by formula II-x: 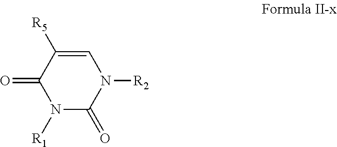
wherein, R₅ is selected from the group consisting of H, amino, halogen, C₁₋₂ alkyl, C₁₋₂ alkoxy, hydroxyl, carboxyl, cyano, alkylamine, heterocycle-substituted alkyl, and (aromatic ring)-substituted alkyl;
R₁ is selected from the group consisting of H or 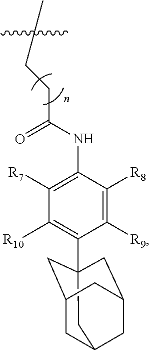
R₂ is selected from the group consisting of H or 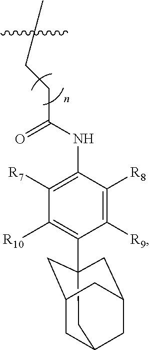
and R₁ and R₂ are not both H; n is an integer selected from 0 to 3; R₇, R₈, R₀, and R₁₀ are each independently selected from the group consisting of H, amino, halogen, C₁₋₄ alkyl, C₁₋₄ alkoxy, halogen, hydroxyl, and carboxyl.

4. The compound according to claim 3, pharmaceutically acceptable salts thereof, and stereoisomers thereof, characterized in that the structure of the compound is as represented by formula II-a or formula II-b: 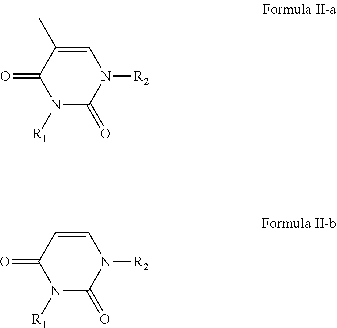
wherein, R₁ is selected from the group consisting of H or 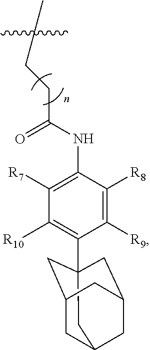
R₂ is selected from the group consisting of H or 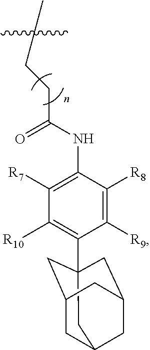
and R₁ and R₂ are not both H; n is an integer selected from 0 to 3; R₇, R₅, R₉, and R₁₀ are each independently selected from the group consisting of H, amino, halogen, C₁₋₄ alkyl, C₁₋₄ alkoxy, halogen, hydroxyl, and carboxyl.

5. The compound according to claim 2, pharmaceutically acceptable salts thereof, and stereoisomers thereof, characterized in that the structure of the compound is as represented by formula II-c or formula II-d: 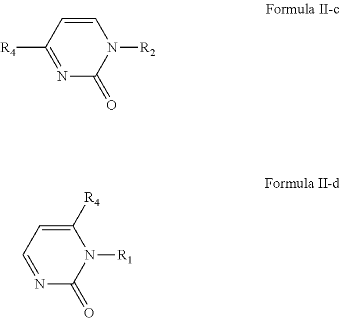
wherein, R₁ is 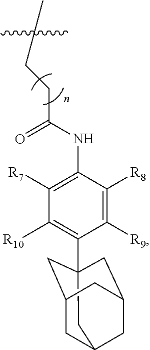
R₂ is 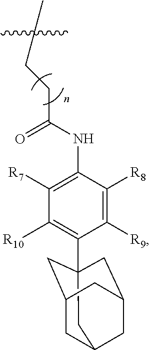
n is an integer selected from 0 to 3; R₇, R₈, R₉, and R₁₀ are each independently selected from the group consisting of H, amino, halogen, C₁₋₄ alkyl, C₁₋₄ alkoxy, halogen, hydroxyl, and carboxyl;
R₄ is selected from the group consisting of H, amino, halogen, C₁₋₄ alkyl, C₁₋₄ alkoxy, hydroxyl, and carboxyl.

6. The compound according to claim 1, pharmaceutically acceptable salts thereof, and stereoisomers thereof, characterized in that the structure of the compound is as represented by formula III-a or formula III-b: 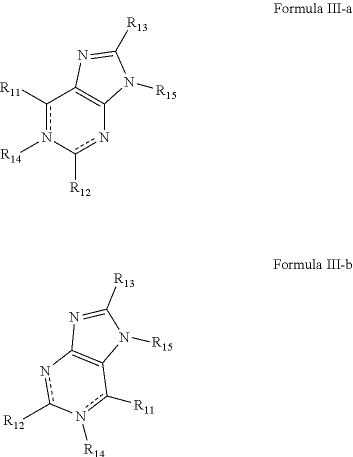
wherein, [symbol] represents [symbol] or [symbol] ;
R₁₄ is selected from the group consisting of absence, H, or 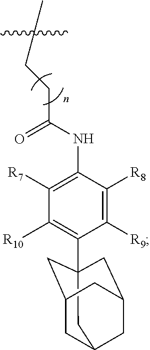
R₁₅ is selected from the group consisting of H or 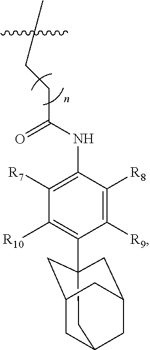
and provided that R₁₄ is absence or H, R₁₅ is not H;
n is an integer selected from 0 to 3; R₇, R₈, R₉, and R₁₀ are each independently selected from the group consisting of H, C₁₋₄ alkyl, C₁₋₄ alkoxy, halogen, hydroxyl, and carboxyl;
R₁₁ is selected from the group consisting of ═O, ═S, H, amino, halogen, C₁₋₄ alkyl, C₁₋₄ alkoxy, hydroxyl, and carboxyl;
R₁₂ is selected from the group consisting of ═O, ═S, H, amino, halogen, C₁₋₄ alkyl, C₁₋₄ alkoxy, hydroxyl, and carboxyl;
R₁₃ is selected from the group consisting of H, amino, halogen, C₁₋₄ alkyl, C₁₋₄ alkoxy, hydroxyl, and carboxyl.

7. The compound according to claim 1, pharmaceutically acceptable salts thereof, and stereoisomers thereof, characterized in that the structure of the compound is as represented by formula III-c or formula III-d: 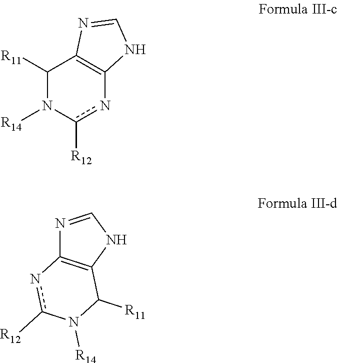
wherein, R₁₄ is 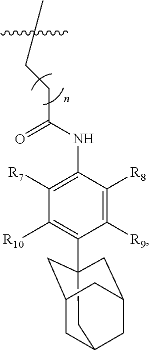
n is an integer selected from 0 to 3; R₇, R₅, R₉, and R₁₀ are each independently selected from the group consisting of H, C₁₋₄ alkyl, C₁₋₄ alkoxy, halogen, hydroxyl, and carboxyl;
R₁₁ is selected from the group consisting of ═O, —S, H, amino, halogen, C₁₋₄ alkyl, C₁₋₄ alkoxy, hydroxyl, and carboxyl;
R₁₂ is selected from the group consisting of ═O, ═S, H, amino, halogen, C₁₋₄ alkyl, C₁₋₄ alkoxy, hydroxyl, and carboxyl.

8. The compound according to claim 1, pharmaceutically acceptable salts thereof, and stereoisomers thereof, characterized in that the compound is selected from the group consisting of:

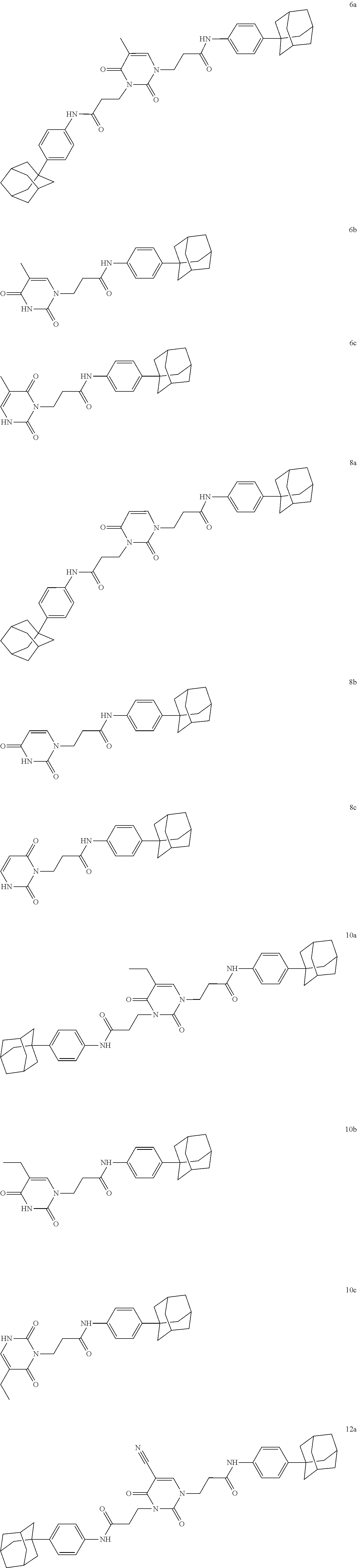

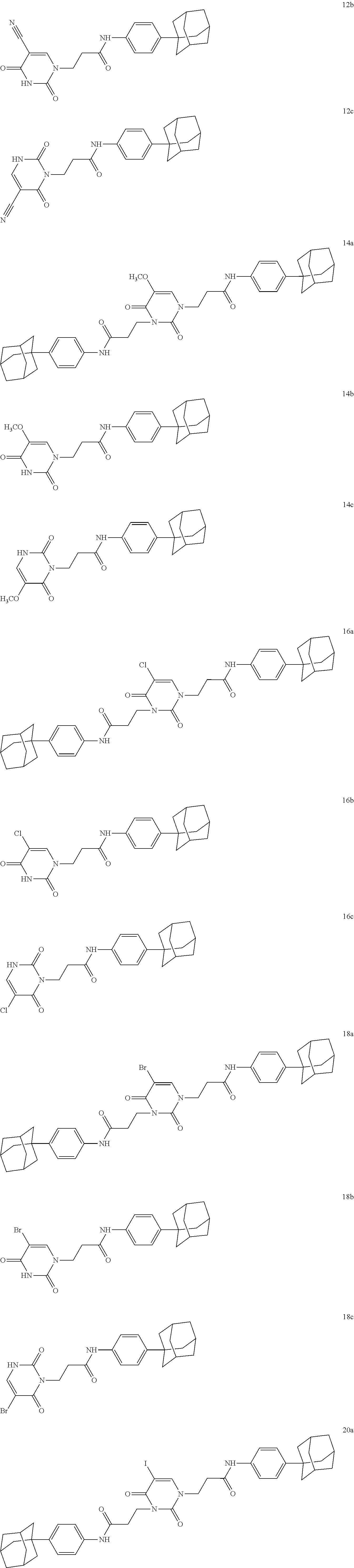

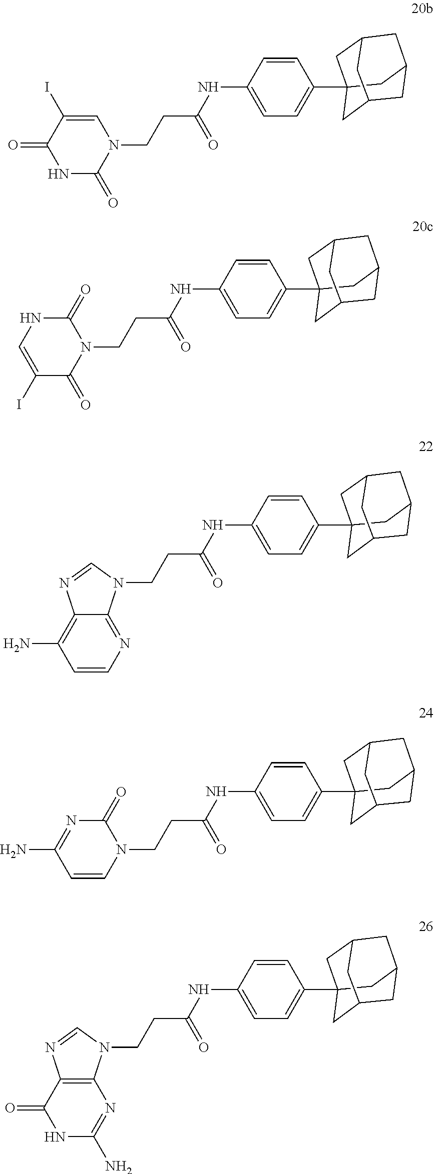 [SMILES: O=C(CCn1cc(I)c(=O)[nH]c1=O)Nc1ccc(C23CC4CC(CC(C4)C2)C3)cc1 ; O=C(CCn1c(=O)[nH]cc(I)c1=O)Nc1ccc(C23CC4CC(CC(C4)C2)C3)cc1 ; Nc1ccnc2c1ncn2CCC(=O)Nc1ccc(C23CC4CC(CC(C4)C2)C3)cc1 ; Nc1ccn(CCC(=O)Nc2ccc(C34CC5CC(CC(C5)C3)C4)cc2)c(=O)n1 ; Nc1nc2c(ncn2CCC(=O)Nc2ccc(C34CC5CC(CC(C5)C3)C4)cc2)c(=O)[nH]1]

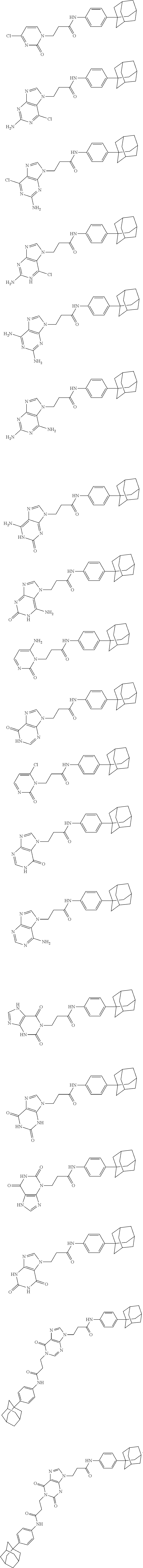

9. An anti-tumor pharmaceutical composition, characterized in that it is a preparation formed by using the compound according to claim 1 as the active ingredient, in combination with pharmaceutically acceptable excipients.

10. The compound according to claim 1 for use in the manufacture of anti-tumor medicaments.

11. The use according to claim 10, characterized in that the tumor is squamous cell carcinoma.

12. The use according to claim 11, characterized in that said squamous cell carcinoma is head and neck squamous cell carcinoma (HNSCC).

13. The use according to claim 12, characterized in that said HNSCC is oral squamous cell carcinoma (OSCC).

14. The use according to claim 13, characterized in that said OSCC is tongue squamous cell carcinoma (TSCC).
